SEQUENCE LISTING (46 sequences)

SEQ ID NO: 1 - protein, 194 aa, Homo sapiens
  MISC_FEATURE (32)..(32): Xaa = Proline or Threonine
MAASLVGKKIVFVTGNAKKLEEVVQILGDKFXCTLVAQKIDLPEYQGEPDEISIQKCQEAVRQVQGPVLVEDTCLCFNALGGLPGPYIKWFLEKLKPEGLHQLLAGFEDKSAYALCTFALSTGDPSQPVRLFRGRTSGRIVAPRGCQDFGWDPCFQPDGYEQTYAEMPKAEKNAVSHRFRALLELQEYFGSLAA

SEQ ID NO: 2 - protein, 177 aa, Homo sapiens
  MISC_FEATURE (15)..(15): Xaa = Prline or Threonine
MAASLVVQILGDKFXCTLVAQKIDLPEYQGEPDEISIQKCQEAVRQVQGPVLVEDTCLCFNALGGLPGPYIKWFLEKLKPEGLHQLLAGFEDKSAYALCTFALSTGDPSQPVRLFRGRTSGRIVAPRGCQDFGWDPCFQPDGYEQTYAEMPKAEKNAVSHRFRALLELQEYFGSLAA

SEQ ID NO: 3 - DNA, 52 bp, Homo sapiens
  misc_feature (27)..(27): M indicates A or C
aactcaccatataacaggggttattcmttatatcctcaaagagtgcactgcc

SEQ ID NO: 4 - DNA, 52 bp, Homo sapiens
  misc_feature (27)..(27): Y indicates C or T
tcagtggccccaagccctcgctcctcyggacccttgcacatgctgttcccag

SEQ ID NO: 5 - DNA, 52 bp, Homo sapiens
  misc_feature (27)..(27): Y indicates C or T
gggcccagggagcaggaaaacacatayacaaacccgcccgctgaccagaaat

SEQ ID NO: 6 - DNA, 52 bp, Homo sapiens
  misc_feature (27)..(27): Y indicates C or T
cagagaggaacaaaataagtttctggyttggctgatctgggtgatcaggtgg

SEQ ID NO: 7 - DNA, 52 bp, Homo sapiens
  misc_feature (27)..(27): Y indicates C or T
ggaccagaaataaagccatacaagtcyaagtaagcataccctttttacttct

SEQ ID NO: 8 - DNA, 52 bp, Homo sapiens
  misc_feature (27)..(27): Y indicates C or T
taagcttgctgtccatgatacagtgayagagcaaaactccggtattataaaa

SEQ ID NO: 9 - DNA, 52 bp, Homo sapiens
  misc_feature (27)..(27): R indicates G or A
tcacagcaaagttgtaatggcctcccrtactgtctgttgtcatcattcagct

SEQ ID NO: 10 - DNA, 52 bp, Homo sapiens
  misc_feature (27)..(27): K indicates G or T
gtggctgtgtggctgaaagactgaatkataaattttgatttttattaattta

SEQ ID NO: 11 - DNA, 52 bp, Homo sapiens; identical to SEQ ID NO: 5
  misc_feature (27)..(27): Y indicates C or T

SEQ ID NO: 12 - DNA, 52 bp, Homo sapiens
  misc_feature (27)..(27): Y indicates C or T
cctgctcccttccttcctattttctayacttgtctcacttcttgacatgttc

SEQ ID NO: 13 - DNA, 52 bp, Homo sapiens
  misc_feature (27)..(27): Y indicates C or T
gcctcctaacaagatggaactagatgytgtcagaggtaagaaaggcacacgc

SEQ ID NO: 14 - DNA, 52 bp, Homo sapiens
  misc_feature (27)..(27): Y indicates C or T
ccagcgtgcgtgtgacactgttaacaygatagggggagactgcttggggaaa

SEQ ID NO: 15 - DNA, 52 bp, Homo sapiens
  misc_feature (27)..(27): M indicates A or C
tttctttcctgcctgttcgtccattamaaatgcaggattcccagggtgccag

SEQ ID NO: 16 - DNA, 52 bp, Homo sapiens
  misc_feature (27)..(27): Y indicates C or T
agctactttagctccacataacccagytattttagctccttttcttgaggtt

SEQ ID NO: 17 - DNA, 52 bp, Homo sapiens
  misc_feature (27)..(27): K indicates G or T
gttgggctcattgttgtcagggtccckggcgaagcggcgaagcatggtccgc

SEQ ID NO: 18 - DNA, 52 bp, Homo sapiens
  misc_feature (27)..(27): M indicates A or C
aacccttacaccccaataccaacatamacagctatcattctcttcccacttc

SEQ ID NO: 19 - DNA, 52 bp, Homo sapiens
  misc_feature (27)..(27): Y indicates C or T
gcctttttggctttgatgcttcttcaygttttgacttttttcaatcacagtt

SEQ ID NO: 20 - DNA, 52 bp, Homo sapiens
  misc_feature (27)..(27): R indicates G or A
atgttttctttccctggggaactcacrcagtatcataggagatggacagctt

SEQ ID NO: 21 - DNA, 52 bp, Homo sapiens
  misc_feature (27)..(27): R indicates G or A
taaaattcagagggaggaaagttttcrtaagtgagacacgaaaggttgagat

SEQ ID NO: 22 - DNA, 52 bp, Homo sapiens
  misc_feature (27)..(27): Y indicates C or T
tttctttaagggctccaactatgcccygccattttgttgcaggcagcataac

SEQ ID NO: 23 - DNA, 52 bp, Homo sapiens
  misc_feature (27)..(27): M indicates A or C
tcgttcagattctaggagataagtttmcatgcactttggtggcacagaaaat

SEQ ID NO: 24 - DNA, 52 bp, Homo sapiens
  misc_feature (27)..(27): M indicates A or C
ttgaccgtatgtctctgttttgttttmtttttaaaagatggttggatttctc

SEQ ID NO: 25 - DNA, 52 bp, Homo sapiens
  misc_feature (27)..(27): Y indicates C or T
ggagcagtggttcacatctgtaactcyggaactttgggaggcccaggtggga

SEQ ID NO: 26 - DNA, 52 bp, Homo sapiens
  misc_feature (27)..(27): S indicates G or C
ctctgtgcctcaggtatctaacagatsaaaggcatgggtttaggacggctaa

SEQ ID NO: 27 - DNA, 52 bp, Homo sapiens
  misc_feature (27)..(27): Y indicates C or T
ggaagaggggaatcccgaatggctgaytgaacaaggatggaaagaaaaccaa

SEQ ID NO: 28 - DNA, 52 bp, Homo sapiens
  misc_feature (27)..(27): Y indicates C or T
ccatcttgcactgctgtcgcttgaagyggtttattaataattattgtttaca

SEQ ID NO: 29 - DNA, 52 bp, Homo sapiens
  misc_feature (27)..(27): R indicates G or A
aagttcctctaggttttcaaatttttrttcttcttccctgttaagatgttca

SEQ ID NO: 30 - DNA, 52 bp, Homo sapiens
  misc_feature (27)..(27): R indicates G or A
gaagaatgaagccaccgaagatagagrgttctgatgacatactagtgcctgc

SEQ ID NO: 31 - DNA, 52 bp, Homo sapiens
  misc_feature (27)..(27): S indicates G or C
aaatctcctgtgatgctggtgttaatstgccctgcattctaacctcagaaca

SEQ ID NO: 32 - DNA, 52 bp, Homo sapiens
  misc_feature (27)..(27): R indicates G or A
atactgtaggcaattataacatgatgrtaatatttgtgaatttaggcatatc

SEQ ID NO: 33 - DNA, 52 bp, Homo sapiens
  misc_feature (27)..(27): Y indicates C or T
cccctagaaacctcaccattcaggtayccagctttgcctttctagccttgga

SEQ ID NO: 34 - DNA, 52 bp, Homo sapiens
  misc_feature (27)..(27): M indicates A or C
ctgtttccagagggagaagacctaaamaaaacagaatttgagcaaagaacac

SEQ ID NO: 35 - DNA, 52 bp, Homo sapiens
  misc_feature (27)..(27): M indicates A or C
aaaaaaaaaaaaatttcaactgatggmaactataagacaaatgatctggttt

SEQ ID NO: 36 - DNA, 52 bp, Homo sapiens
  misc_feature (27)..(27): Y indicates C or T
cgtgttgcacactcaaaataatcatgyttacagagacttcatgagctagtca

SEQ ID NO: 37 - DNA, 52 bp, Homo sapiens
  misc_feature (27)..(27): R indicates G or A
tttcctagagctcgtattttccatacrttcagttattacaacattcacttgt

SEQ ID NO: 38 - DNA, 52 bp, Homo sapiens
  misc_feature (27)..(27): R indicates G or A
gaaggaaaaaggaagcaatgtgtttcrgttcaaacatttcctagctggcttt

SEQ ID NO: 39 - DNA, 52 bp, Homo sapiens
  misc_feature (27)..(27): Y indicates C or T
ccttcctccctgtcctcttggctgaayttttcctctcccctactttctgctc

SEQ ID NO: 40 - DNA, 52 bp, Homo sapiens
  misc_feature (27)..(27): Y indicates C or T
cgcactgccaggcccatagagaagcaygcctggtaagcagggctggcgtgtg

SEQ ID NO: 41 - DNA, 52 bp, Homo sapiens
  misc_feature (27)..(27): R indicates G or A
ctcagaacacacaggcaagagagtgcrtccggacccatccaggtcagcagac

SEQ ID NO: 42 - DNA, 52 bp, Homo sapiens
  misc_feature (27)..(27): R indicates G or A
tatcccttcatctagcacagtgactgrcatttactagacattcaacaaggat

SEQ ID NO: 43 - DNA, 52 bp, Homo sapiens
  misc_feature (27)..(27): M indicates A or C
tctgttttggcctcaaagggttcagamtaaaaggggcttttctcttgtgaga

SEQ ID NO: 44 - DNA, 52 bp, Homo sapiens
  misc_feature (27)..(27): Y indicates C or T
tgtcccaaattagactagcaggaactycttaagctacttcccatatcctttt

SEQ ID NO: 45 - DNA, 52 bp, Homo sapiens
  misc_feature (27)..(27): Y indicates C or T
ttgttaagatgtttgattaatgtcttyttctccactaccctataaactttat

SEQ ID NO: 46 - DNA, 52 bp, Homo sapiens
  misc_feature (27)..(27): R indicates G or A
aaaacaacaactaacagctccacttgrtgtcaaagttcaattctattgcccc

(19) United States
(12) Patent Application Publication
(10) Pub. No.: US 2012/0282224 A1
(43) Pub. Date: Nov. 8, 2012

(54) MARKERS ASSOCIATED WITH RIBAVIRIN-INDUCED ANEMIA

(76) Inventors: Janice K. Albrecht, Winter Park, FL (US); Clifford A. Brass, New York, NY (US); Jacques Fellay, St. Maurice (CN); Dongliang Ge, Cary, NC (US); David B. Goldstein, Durham, NC (US); Curtis Gumbs, Durham, NC (US); John G. McHutchinson, Chapel Hill, NC (US); Ping Qiu, Edison, NJ (US); Kevin Shianna, Durham, NC (US); Alexander J. Thompson, Victoria (AU); Thomas J. Urban, Durham, NC (US)

(21) Appl. No.: 13/508,605

(22) PCT Filed: Nov. 5, 2010
(86) PCT No.: PCT/US10/55570

§ 371 Date: Jul. 19, 2012

(30) Foreign Application Priority Data
Nov. 9, 2009 (US) 61259487

(51) Int. Cl. C12Q 1/68; A61K 38/07; A61K 38/21; A61K 38/06; A61K 31/7056; A61P 31/14

(52) U.S. Cl. 424/85.7; 514/43; 435/6.11; 514/4.3

(57) ABSTRACT

The present invention provides genetic markers and biomarkers that are associated with anemia induced by ribavirin therapy. The genetic markers are located in the ITPA gene and elsewhere on human chromosome 20 and the biomarkers are low ITPA activity phenotypes. These markers of ribavirin-induced anemia are useful, inter alia, to identify patients who are least likely to develop anemia upon treatment with ribavirin pharmaceutical compositions and drug products, in methods of treating patients having a disease susceptible to treatment with ribavirin, and in methods for selecting the most appropriate therapy for such patients.

### FIELD OF THE INVENTION

The present invention relates to markers that are associated with adverse effects of ribavirin therapy, and in particular to genetic polymorphisms and biomarkers that are associated with ribavirin-induced anemia.

### BACKGROUND OF THE INVENTION

Identification of any publication in this section or any section of this application is not an admission that such publication is prior art to the present invention.

Ribavirin (1-[(2R,3R,4S,5R)-3,4-dihydroxy-5-(hydroxymethyl)oxolan-2-yl]-1H-1,2,4-triazole-3-carboxamide, also known as 1-(β-D-Ribofuranosyl)-1H-1,2,4-triazole-3-carboxamide) is a nucleoside analogue with broad spectrum antiviral activity. The primary clinical use of ribavirin (RBV) has been in combination with an interferon alpha for the treatment of hepatitis C virus (HCV) infections, with the current standard of care combining RBV with a pegylated interferon alpha (PegIFN) (either peginterferon alfa-2a, marketed by Hoffman-La Roche (Nutley, N.J.) under the trade name PEGASYS®, or peginterferon alfa-2b, marketed by Schering-Plough (Kenilworth, N.J.) under the trade name PegIntron®). Peg-IFN/RBV combination therapy is associated with a range of treatment-limiting adverse effects.

One of these adverse effects is RBV-induced anemia, which affects a majority of patients. RBV-induced anemia, which typically begins during the first 4 weeks of therapy, is due to two mechanisms: hemolysis of erythrocytes (i.e., hemolytic anemia) and suppression of erythropoiesis (by concurrent use of interferon). Hemolytic anemia affects nearly all patients treated with ribavirin, but the extent of hemoglobin reduction can vary considerably among individuals.

The mechanism of RBV-induced hemolytic anemia has been recently described (De Franceschi, L. et al., Hepatol. 31:997-1004 (2000); Line, C-C., et al., J. Clin. Pharmacal. 44:265-275 (2004). Plasma ribavirin enters cells as a prodrug and is converted into ribavirin 5′-monophosphate (RMP), -diphosphate (RDP) and -triphosphate (RTP), leading to depletion of adenosine triphosphate (ATP). Because erythrocytes lack the phosphatases needed to hydrolyze ribavirin phosphates, they accumulate, with RTP concentrations reaching 60-fold greater levels in erythrocytes than in plasma. The combined accumulation of ribavirin phosphates and relative ATP deficiency makes the erythrocyte highly susceptible to oxidative stress by the reticuloendothelial system, resulting in extravascular hemolysis.

In clinical trials of Peg-IFN/RBV combination therapy, hemoglobin (Hb) levels decreased by an average of 2-3 g/dL. Hb levels in greater than 50% of patients decreased to <12 g/dL, with moderate anemia (Hb<11 g/dL) occurring in about 30% of patients and severe anemia (Hb<10 g/dL) that required ribavirin dose reduction occurring in up to 15% of patients. Manns, M. P., et al., Lancet 358:958-965 (2001). Reduction in RBV dose, however, can have a negative impact on the efficacy of Peg-IFN/RBV combination therapy (McHutchinson, J. G., et al., Gastroenterol. 123:1061-1069 (2002); Shiffman, M. L. et al., Gastroenterol. 126:1015-1023 (2004); Hadziyannis, S. J. et al., Ann Intern Med 140:346-355 (2004)). Moreover, even moderate anemia impairs the quality of life, may alter adherence to treatment and result in premature discontinuation of therapy.

To minimize these undesired outcomes, agents that counteract treatment-induced anemia, such as recombinant human erythropoietin (epoetin alfa), are frequently used as adjuvant therapy. However, such adjuvant therapy adds complexity and cost to an already complicated and expensive treatment regimen.

Thus, a need exists to identify patients who are at risk for moderate or severe RBV-induced anemia and thus who may benefit the most from treatment regimens designed to maintain adequate ribavirin during therapy. Several patient baseline characteristics have been identified as prognostic factors for RBV-induced anemia, including gender, ribavirin dose per kilogram, baseline (pre-therapy) hemoglobin concentration, age, cirrhosis and impaired renal function (see, e.g., Sulkowski, M. S. et al., J. Viral Hepatol. 11(3): 243-250 (2004). The present invention adds to this list of prognostic factors by providing genetic markers that are correlated with RBV-induced anemia.

### SUMMARY OF THE INVENTION

The present invention is based on a retrospective, genome-wide analysis study (GWAS) of HCV patients of three ethnic groups (European American, African American, and Hispanic) treated with RBV combined with peginterferon alfa-2b or peginterferon alfa-2a which resulted in the identification of associations between treatment-induced hemoglobin (Hb) reduction and single nucleotide polymorphisms (SNPs) on chromosome 20 and chromosome 10.

One of these associated SNPs is an A/C polymorphism located in the p13 region of chromosome 20 (20p13) and identified as rs6051702 in the NCBI SNP Database. Individuals who are heterozygous or homozygous for the C allele were significantly less likely than individuals homozygous for the A allele to experience a decrease in hemoglobin (Hb) of at least 3 g/dl during the first 4 weeks of treatment.

The inventors found that in European Americans the protective C allele of rs6051702 is in linkage disequilibrium (LD) with two variants in the gene encoding inosine triphosphatase (ITPA) that have been causally linked to reduced ITPA activity: a 94C>A missense variant in exon 2 that results in the substitution of threonine for proline (P32T) (rs1127354) and may impair association of ITPA monomers into a dimeric enzyme or cause missplicing of exons 2 and 3; and a splicing-altering SNP located in the second intron (IVS2+21A>C; rs7270101) that results in missplicing of exon 3. Sumi, S, et al., Hum Genet. 111:360-367 (2002); Cao, H. et al., Hum Genet. 47:620-622 (2002); Arenas, M. et al., Biochim Biophys Acta 1772:96-102 (2007). The rs1127354 A allele has been found in all ethnic populations, but its frequency varies significantly between populations, from 1-2% in Central- and South American populations up to 11-19% in Asian populations (Marsh S. et al., J. Hum. Genet. 49:579-581 (2004)). In Caucasian, African-Americans and African populations, the rs1127354 A allele frequency is 5-7%. The allele frequency of the rs7270101 C allele is approximately 13% in Caucasian populations and was not observed in a Japanese population (Maeda T., et al., Mol. Genet. Metab. 85:271-279 (2005)).

To test the possibility that these low activity ITPA variants may confer protection against RBV-induced anemia, the inventors genotyped the rs1127354 and rs7270101 SNPs in the HCV patient cohort and found that these ITPA SNPs entirely explain the association signal observed for rs6051702. Based on these findings, the inventors herein believe that inosine triphosphatase deficiency protects against RBV-induced hemolytic anemia.

Inosine triphosphatase deficiency is a red cell enzymopathy characterized by the accumulation of inosine triphosphate (ITP) in erythrocytes and associated with adverse responses to the thiopurine drugs azathiopurine and 6-mercaptopurine (Bierau, J. et al., Pharmacogenomics 8(9):1221-1228 (2007). Since the mechanism of RBV-induced hemolytic anemia involves the accumulation of RBV-TP in red blood cells, the protective effect of inosine triphosphatase deficiency against RBV-induced anemia may be explained by competition of ITP with RBV-TP in the cellular processes affected by RBV-TP, thereby protecting cells from the lytic effects of RBV-TP.

Thus, the inventors believe that the identification of individuals who are most likely to have clinically significant RBV-induced anemia (e.g., a Hb decrease of ≧3 g/dL or Hb≦10 g/dL) may be accomplished by testing for the presence or absence of any of the following: the normal ITPA activity allele of any SNP in the ITPA gene that is associated with ITPA deficiency (e.g., the anemia-associated alleles of the rs1127354 or rs7270101 SNPs), any of the SNPs in the GWAS described herein, and the allele at any other SNP in the 20p13 region that is in high LD with a normal ITPA activity allele. These SNPs are described in Table 1 below, which lists the polymorphic site (PS) where the SNP is located, identified with the NCBI SNP Database designation, the alternative alleles that are found at the PS, the allele that is associated with RBV-induced anemia (referred to herein as the “anemia allele”), and the heterozygous and homozygous genotypes comprising this allele, which are referred to herein as RIA (Ribavirin-Induced Anemia) genetic markers. All of the SNPs in Table 1 are located in chromosome 20 except for rs10159477, which is in the hexokinase gene on chromosome 10.

TABLE 1
Genetic Markers of Ribavirin-Induced Anemia (RIA)

| Experimental | | | Anemia | Heterozygous | Homozygous |
|---|---|---|---|---|---|
| Evidence^{a} | PS | SNP | Allele | RIA Marker | RIA Marker |
| 1 | rs6051702 | A/C | A | A/C genotype | A/A genotype |
| 1 | rs3810560 | A/G | A | A/G genotype | A/A genotype |
| 1 | rs11697114 | T/C | T | T/C genotype | T/T genotype |
| 1 | rs3310 | T/C | C | T/C genotype | C/C genotype |
| 1 | rs965469 | T/C | T | T/C genotype | T/T genotype |
| 1 | rs6051762 | T/C | T | T/C genotype | T/T genotype |
| 1 | rs6051841 | T/C | T | T/C genotype | T/T genotype |
| 1 | rs6051693 | T/G | T | T/G genotype | T/T genotype |
| 1 | rs6115892 | T/C | C | T/C genotype | C/C genotype |
| 1 | rs6115865 | T/C | C | T/C genotype | C/C genotype |
| 1 | rs6051855 | T/C | T | T/C genotype | T/T genotype |
| 1 | rs11697620 | A/G | A | A/G genotype | A/A genotype |
| 1 | rs2295547 | A/C | C | A/C genotype | C/C genotype |
| 1 | rs8120592 | T/C | C | T/C genotype | C/C genotype |
| 1 | rs3827075 | A/C | C | A/C genotype | C/C genotype |
| 1 | rs2326084 | A/C | A | A/C genotype | A/A genotype |
| 1 | rs1207 | T/C | T | T/C genotype | T/T genotype |
| 1 | rs2295545 | T/C | C | T/C genotype | C/C genotype |
| 1 | rs10159477 | T/C | T | T/C genotype | T/T genotype |
| 1 | rs6076519 | T/C | C | T/C genotype | C/C genotype |
| 1 | rs6051689 | A/G | G | A/G genotype | G/G genotype |
| 2 | rs1127354 | C/A | C | A/C genotype | C/C genotype |
| 2 | rs7270101 | A/C | A | A/C genotype | A/A genotype |
| 3 | rs7274193 | C/T | C | C/T genotype | C/C genotype |
| 3 | rs2236094 | G/C | G | G/C genotype | G/G genotype |
| 3 | rs6051708 | T/C | T | T/C genotype | T/T genotype |
| 3 | rs6051790 | C/T | C | C/T genotype | C/C genotype |
| 3 | rs6037553 | A/G | A | A/G genotype | A/A genotype |
| 3 | rs6139064 | G/T | G | G/T genotype | G/G genotype |
| 3 | rs4611719 | A/G | A | A/G genotype | A/A genotype |
| 3 | rs2236123 | C/G | C | C/G genotype | C/C genotype |
| 3 | rs2236118 | G/A | G | GIA genotype | G/G genotype |
| 3 | rs2236122 | C/T | C | C/T genotype | C/C genotype |
| 3 | rs2236104 | C/A | C | C/A genotype | C/C genotype |
| 3 | rs6037567 | C/A | C | C/A genotype | C/C genotype |
| 3 | rs6051716 | C/T | C | C/T genotype | C/C genotype |
| 3 | rs6051807 | A/G | A | A/G genotype | A/A genotype |
| 3 | rs6051753 | G/A | G | G/A genotype | G/G genotype |
| 3 | rs6051764 | C/T | C | C/T genotype | C/C genotype |
| 3 | rs1040726 | C/T | C | CIT genotype | C/C genotype |
| 3 | rs2281500 | G/A | G | G/A genotype | G/G genotype |
| 3 | rs6037554 | G/A | G | G/A genotype | G/G genotype |
| 3 | rs2236089 | C/A | C | C/A genotype | C/C genotype |
| 3 | rs7270135 | T/C | T | T/C genotype | T/T genotype |
| 3 | rs6037560 | T/C | T | T/C genotype | T/T genotype |
| 3 | rs6051713 | A/G | A | A/G genotype | A/A genotype |

^{a}The number refers to the experimental evidence for association with RBV-induced anemia: 1 means the SNP was identified in the GWAS study described herein; 2 means the SNP is functionally associated with ITPA activity and independently associated with RBV-induced anemia; and 3 means the SNP is in LD with the listed ITPA SNPs.

The inventors herein contemplate that testing individuals for (1) the presence of one or more of the RIA genetic markers in Table 1 and/or (2) for normal erythrocyte ITPA activity will be useful to identify individuals most likely to experience anemia in response to ribavirin therapy for a disease susceptible to treatment with ribavirin. These RIA markers (genetic SNP markers and ITPA activity biomarkers) should also identify individuals most likely to experience anemia in response to any ribavirin analogue (e.g., taribavirin) that is phosphorylated in erythrocytes to generate a triphosphate that is structurally similar to RBV-TP. Ribavirin and such structural analogues are collectively referred to herein as ribavirin compounds.

Accordingly, in one embodiment, the invention provides a pharmaceutical composition comprising a ribavirin compound for treating an individual having a disease susceptible to treatment with the ribavirin compound and a negative test for at least one RIA marker.

In another embodiment, the invention provides the use of a ribavirin compound in the manufacture of a medicament for treating an individual having a disease susceptible to treatment with the ribavirin compound and a negative test for at least one RIA marker.

In yet another embodiment, the invention provides a drug product which comprises a ribavirin pharmaceutical composition and prescribing information which includes a pharmacogenetic indication for which the pharmaceutical composition is recommended. The pharmacogenetic indication includes two components: a disease susceptible to treatment with the ribavirin compound in the pharmaceutical composition and patients who have the disease and who are genetically defined by lacking at least one RIA marker.

The invention also provides a method of testing an individual for the presence or absence of at least one RIA marker, the method comprising obtaining a nucleic acid sample from the individual and assaying the sample to determine the individual's genotype for at least one of the polymorphic sites in Table 1.

In another embodiment, the invention provides a method of testing an individual for the presence of an RIA marker, the method comprising obtaining a biological sample from the individual and assaying the biological sample for the presence of ITPA with proline at amino acid position 32 (ITPA-Pro32). In some embodiments, the assaying step comprises contacting the biological sample with a monoclonal antibody that specifically binds to ITPA-Pro32 (i.e., does not bind to ITPA-Thr32). In other embodiments, the assaying step comprises contacting the biological sample with each of a monoclonal antibody that specifically binds to ITPA-Pro32 and a monoclonal antibody that specifically binds to ITPA-Thr32 (i.e., does not bind to ITPA-Pro32).

In yet another embodiment, the invention provides a method of predicting whether an individual is at risk for severe anemia (Hb<10 g/dL) if treated with a ribavirin compound, the method comprising obtaining an erythrocyte sample from the individual, measuring the ITPA activity in the sample and comparing the ITPA activity to a standard (e.g., the range for normal ITPA activity), wherein if the measured ITPA activity is lower than the standard then the prediction is that the individual is not likely to experience severe anemia upon treatment with the ribavirin compound, and if the measured ITPA activity is not lower than the standard (e.g., within or higher than the normal range) then the prediction is that the individual is likely to experience severe anemia upon treatment with the ribavirin compound.

In some embodiments, the method of testing individuals for the presence or absence of an RIA marker further comprises generating a test report that indicates the individual's genotype for the assayed polymorphic site and optionally providing the test report to the individual or to a physician who is treating the individual for a disease susceptible to treatment with the ribavirin compound.

In another aspect, the invention provides a kit for detecting an RIA marker in a nucleic acid sample. The kit comprises a set of one or more oligonucleotides designed for identifying each of the alleles at the polymorphic site in the RIA marker. In some embodiments, the nucleic acid sample is from a patient having a disease susceptible to treatment with a ribavirin compound. In some preferred embodiments, the disease is a chronic HCV infection. In other preferred embodiments, the ribavirin compound is ribavirin or taribavirin.

In a still further embodiment, the invention provides a method of selecting a therapy for treating an individual having a disease susceptible to treatment with a ribavirin compound, comprising obtaining the individual's genotype for the presence of at least one RIA marker and selecting a therapy based on the obtained genotype. In some embodiments, if the individual has the RIA marker, the selected therapy comprises administering the ribavirin compound in combination with an agent that counteracts ribavirin-induced anemia. In other embodiments, the selected therapy for an individual having an RIA marker comprises treatment with a dose of the ribavirin compound that is lower than recommended for the disease or excludes treatment with the ribavirin compound. If the individual lacks the RIA marker, the selected therapy in some embodiments comprises administering the ribavirin compound at either the dose recommended for the disease or at a higher than recommended dose and monitoring the individual for anemia.

The invention also provides a screening method for selecting individuals for initial treatment or continued treatment with a ribavirin compound from a group of individuals having a disease susceptible to treatment with the ribavirin compound. This screening method comprises testing each member of the disease group for the presence of at least one RIA marker and excluding from treatment all individuals testing positive for the RIA marker.

In each of the above embodiments that employ an RIA genetic marker, the marker is any of the heterozygous and homozygous RIA markers shown in Table 1. In preferred embodiments, the RIA marker is one of the homozygous RIA markers. In one preferred embodiment, the RIA marker is a C/C genotype at rs1127354 or an A/A genotype at rs7270101. In another preferred embodiment, the RIA marker is an A/A genotype at the rs6051702 PS if the individual is of Caucasian ethnicity or an A/A genotype at rs3810560 PS if the individual is of African ethnicity or a TIT genotype at rs11697114 if the individual is of Hispanic ethnicity. In other embodiments, the prediction of severe anemia induced by treatment with a ribavirin compound is based on the presence of an RIA marker for each of at least two PS in Table 1, and in preferred embodiments, the two PS are rs1127354 and rs7270101.

In some embodiments of any of the above compositions and methods in which the disease susceptible to treatment with a ribavirin compound is a chronic HCV infection, the chronic HCV infection is a high baseline viral load infection with an HCV genotype selected from the group consisting of genotype 1 (G1 HCV), genotype 3 (G3 HCV) or genotype 4 (G4 HCV).

In all of the above embodiments, a positive test for an RIA marker may be used in combination with the presence of one or more other predictors of RBV-induced anemia to identify patients who are likely to experience severe anemia upon treatment with a ribavirin compound.

### BRIEF DESCRIPTION OF THE DRAWINGS

FIG. 1 illustrates reference amino acid sequences for two human ITPAA isoforms encoded by transcripts of different length, with the longer transcript encoding the 194 amino acid isoform a shown in FIG. 1A (SEQ ID NO:1) and the shorter transcript encoding the 177 amino acid isoform b shown in FIG. 1B (SEQ ID NO:2), with the location of variant amino acid positions indicated by a bold letter in the reference sequence and the identity of the variant (less frequent) allele indicated by a bold letter below the variant amino acid position.

FIG. 2 illustrates the results of single-marker genotype trend tests for significant determinants of treatment-induced reduction in hemoglobin in a combined group of European American, African American and Hispanic patients chronically infected with HCV genotype 1 and treated with Peg-IFN alfa-2a or 2b/ribavirin combination therapy for 4 weeks. The top and middle graphs show the p values [−log(P)] of all genotyped SNPs from the genome wide and chromosome 20p13 region, respectively, with the tallest 10 vertical lines indicating the SNPs that showed genome-wide significant association with reduction in hemoglobin. The bottom graph shows the locations and structures for the ITPA gene and surrounding genes in the 20p13 region.

FIG. 3 illustrates the association between genotype at the rs6051702 polymorphic site (CC, AC or AA) (X-axis) and the percentage of patients with each genotype who presented a ≧3 g/dL decrease in Hb levels (Y-axis) in different patient groups chronically infected with HCV genotype 1 and treated with Peg-IFN/RBV combination therapy, with N representing the total number of subjects with each genotype in the indicated patient group. Further details are in the Examples.

FIG. 4 illustrates the proportion of patients chronically infected with HCV genotype 1 and treated with Peg-IFN/RBV combination therapy who experienced moderate anemia (decrease in Hb of ≧3 g/dL, dotted) or severe anemia (Hb≦10 g/dL, cross-hatch) as a function of genotype for two ITPA SNPs associated with ITPA activity (rs1127354, upper left graph and rs7270101, upper right graph) or predicted ITPA deficiency (lower graph), with +++ indicating very low residual activity, ++ indicating 30% of normal activity and + indicating 60% of normal activity and N indicating the number of patients with each ITPA genotype or predicted phenotype. Further details are in the Examples.

### DETAILED DESCRIPTION OF THE INVENTION

### I. General

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

### II. Definitions

So that the invention may be more readily understood, certain technical and scientific terms are specifically defined below. Unless specifically defined elsewhere in this document, all other technical and scientific terms used herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“About” when used to modify a numerically defined parameter, e.g., the dosage for a therapeutic agent discussed herein, or the length of treatment time, means that the parameter may vary by as much as 10% above or below the stated numerical value for that parameter. For example, a dose of about 800 mg of ribavirin used in the treatment of HCV patients could vary between 720 mg and 880 mg.

“Allele” is a particular form of a gene or other genetic locus, distinguished from other forms by its particular nucleotide sequence, the term allele also includes one of the alternative polymorphisms (e.g., a SNP) found at a polymorphic site.

“Beneficial result” means a desired clinical result of treatment with a ribavirin compound, including but not limited to: alleviation of one or more disease symptoms, diminishment of extent of disease (e.g., reduction in viral load), stabilized (i.e., not worsening) state of disease, slowing of disease progression, amelioration or palliation of a disease state, prolonging survival (as compared to expected survival if not treated), relapse-free survival, remission (whether partial or total) and cure (i.e., elimination of the disease).

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

“Individual” or “animal” or “patient” or “mammal,” is meant any subject, particularly a mammalian subject, for whom any of the claimed compositions and methods is needed or may be beneficial. In preferred embodiments, the individual is a human. In more preferred embodiments, the individual is an adult human, i.e., at least 18 years of age.

“Inosine Triphosphatase” (ITPA or ITPase) or “Inosine Triphosphate Pyrophosphohydrolase” means a polypeptide comprising amino acids of SEQ ID NO:1 (Isoform a, NCBI Reference Sequence NP_{—}258412.1, GI:15626999) or SEQ ID NO:2 (Isoform b, NCBI Reference Sequence NP_{—}852470.1; GI:31657144). In ITPA isoform a, which is encoded by a transcript containing an open reading frame of 585 nucleotides, the proline or threonine allelic variant is located at amino acid position 32. This allelic variant is located at position 15 of ITPA isoform b, which has 177 amino acids and is encoded by a shorter transcript that uses an alternate in-frame splice site in the 5′ coding region. Thus an ITPAa Thr polypeptide is an ITPase isoform a having threonine at amino acid position 32 of SEQ ID NO:1 and an ITPAb Thr polypeptide is an ITPase isoform b having threonine at amino acid position 15 of SEQ ID NO:2. Similarly, an ITPAa Pro polypeptide is an ITPase isoform a having proline at amino acid position 32 of SEQ ID NO:1 and an ITPAb Pro polypeptide is an ITPase isoform b having proline at amino acid position 15 of SEQ ID NO:2.

“Isolated” is typically used to reflect the purification status of a biological molecule such as RNA, DNA, oligonucleotide, or protein, and in such context means the molecule is substantially free of other biological molecules such as nucleic acids, proteins, lipids, carbohydrates, or other material such as cellular debris and growth media. Generally, the term “isolated” is not intended to refer to a complete absence of other biological molecules or material or to an absence of water, buffers, or salts, unless they are present in amounts that substantially interfere with the methods of the present invention.

“ITPA activity” refers to the rate of conversion of inosine triphosphate (ITP) to inosine monophosphate (IMP), and is expressed as micromole (mole) of IMP formed per gram of hemoglobin per hour [μmole/(g Hb·h)]. The “normal ITPA activity” is an activity that is observed for a population of healthy individuals of similar ethnic origin, e.g., Caucasians, Asians, African Americans, Hispanics who have a C/C genotype at the rs1127354 PS and an A/A genotype at the rs7270101 PS. For Caucasian females, a normal ITPA activity is any value within the range of 133.9-362.0 μmole/(g Hb·h) and for Caucasian males a normal ITPA activity is any value within the range of 154.3-408.3 μmole/(g Hb·h).

“Locus” refers to a location on a chromosome or DNA molecule corresponding to a gene, a physical feature such as a polymorphic site, or a location associated with a phenotypic feature.

“Nucleotide pair” is the set of two nucleotides (which may be the same or different) found at a polymorphic site on the two copies of a chromosome from an individual.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a locus; such oligonucleotides are referred to as allele-specific probes. If the locus is a PS comprising a SNP, the complementary allele for that SNP can occur at any position within an allele-specific probe. Other oligonucleotides useful in practicing the invention specifically hybridize to a target region adjacent to a PS with their 3′ terminus located one to less than or equal to about 10 nucleotides from the PS, preferably about 5 nucleotides. Such oligonucleotides hybridizing adjacent to a PS are useful in polymerase-mediated primer extension methods and are referred to herein as “primer-extension oligonucleotides”. In a preferred embodiment, the 3-terminus of a primer-extension oligonucleotide is a deoxynucleotide complementary to the nucleotide located immediately adjacent to the PS.

“Parenteral administration” means an intravenous, subcutaneous, or intramuscular injection.

“Pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“Polymorphic site” or “PS” refers to the position in a genetic locus or gene at which a polymorphism is found, e.g., single nucleotide polymorphism (SNP), restriction fragment length polymorphism (RFLP), variable number of tandem repeat (VNTR), dinucleotide repeat, trinucleotide repeat, tetranucleotide repeat, simple sequence repeat, insertion element such as Alu, and deletion or insertion of one or more nucleotides). A PS is usually preceded by and followed by highly conserved sequences in the population of interest and thus the location of a PS is typically made in reference to a consensus nucleic acid sequence of thirty to sixty nucleotides that bracket the PS, which in the case of a SNP is commonly referred to as the “SNP context sequence”. The location of the PS may also be identified by its location in a consensus or reference sequence relative to the initiation codon (ATG) for protein translation. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence (or with respect to an initiation codon in such a sequence) is merely for convenience and that any specifically enumerated nucleotide position literally includes whatever nucleotide position the same PS is actually located at in the same locus in any individual being tested for the presence or absence of a genetic marker of the invention using any of the genotyping methods described herein or other genotyping methods well-known in the art.

“Ribavirin response” means a desired clinical result of treatment with a ribavirin compound, including but not limited to: alleviation of one or more disease symptoms, diminishment of extent of disease, stabilized (i.e., not worsening) state of disease, slowing of disease progression, amelioration or palliation of a disease state, prolonging survival (as compared to expected survival if not treated), relapse-free survival, remission (whether partial or total) and cure (i.e., elimination of the disease).

“Ribavirin treatment naïve” means that the individual or patient who is to be treated or tested according to any of the embodiments described herein has not been previously treated with any ribavirin compound, including any experimental or approved ribavirin drug product.

“Treat” or “Treating” means to administer a therapeutic agent, such as a composition containing any of the ribavirin compounds described herein, internally or externally to an individual in need of the therapeutic agent. Individuals in need of the agent include individuals who have been diagnosed as having, or at risk of developing, a condition or disorder susceptible to treatment with the agent, as well as individuals who have, or are at risk of developing, one or more adverse effects of treatment with a first therapeutic agent that are susceptible to alleviation with a second therapeutic agent. Typically, the therapeutic agent is administered in a therapeutically effective amount, which means an amount effective to produce one or more beneficial results. The therapeutically effective amount of a particular agent may vary according to factors such as the disease state, age, and weight of the patient being treated, and the sensitivity of the patient, e.g., ability to respond, to the therapeutic agent. Whether a beneficial or clinical result has been achieved can be assessed by any clinical measurement typically used by physicians or other skilled healthcare providers to assess the presence, severity or progression status of the targeted disease, symptom or adverse effect. Typically, a therapeutically effective amount of an agent will result in an improvement in the relevant clinical measurement(s) over the baseline status, or over the expected status if not treated, of at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. While an embodiment of the present invention (e.g., a treatment method or article of manufacture) may not achieve the desired clinical benefit or result in every patient, it should do so in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Viral load” in the context of treating chronic HCV infection means the quantity of HCV RNA in the serum of a patient (also referred to in the art and herein as serum HCV RNA and HCV viral load). The viral load is preferably measured using a quantitative RT-PCR assay that is generally accepted in the art as providing a reliable result. More preferably, the RT-PCR assay used to measure an HCV viral load has a lower limit of quantitation (LLQ) of about 29 international units/mL (IU/mL) or less. Quantifying a patient's HCV viral load at baseline and at various time points during treatment with antiviral therapy is useful to classify whether the patient has a high baseline viral load, as defined herein, and to assign the patient to a viral response phenotype, including any one of the viral response phenotypes described herein.

“Baseline viral load” means the serum HCV RNA level prior to initiation of therapy with one or more antiviral agents. A “high baseline viral load” means a quantity of HCV RNA that is generally understood in the art as classifying a patient as having a difficult to treat chronic HCV viral infection. Two baseline viral load values that have been used to classify patients as difficult to treat in the context of indirect peginterferon alfa/ribavirin therapy are >600,000 IU/ml and >800,000 IU/ml. Recently, a viral load used to classify patients as being difficult to treat is >400,000 IU/ml.

“Undetectable HCV RNA” means that HCV RNA was not detected using an RT-PCR assay with a lower limit of detection (LLD) of about 10 IU/ml or less or any other assay that employs different methodology but is generally accepted in the art as providing an equivalent or similar sensitivity.

“Viral response” in the context of treating chronic HCV infection means a reduction in the level of serum HCV RNA after initiation of antiviral therapy.

In some embodiments, the antiviral therapy comprises a ribavirin compound and an interferon alpha. Combination therapy that includes an interferon alpha is frequently referred to in the art as interferon-alpha based therapy. In other embodiments, the viral response being measured is response to antiviral therapy that does not include an interferon alpha. Preferred viral response phenotypes are rapid viral response (RVR), early viral response (EVR), end of treatment response (ETR), sustained viral response (SVR), slow response, null response, nonresponse (NR) and relapse. The definitions and time points for assessing these response phenotypes are described below. In some embodiments, the HCV treatment comprises a lead-in period of indirect antiviral therapy, such as combination peginterferon alpha/ribavirin therapy, followed by “direct antiviral therapy”, which as used herein means that the therapy comprises administration of at least one direct antiviral agent, such as an HCV protease inhibitor, optionally in combination with one or more indirect antiviral agents, such as a pegylated interferon and ribavirin. In such multi-phase treatment regimens, the viral response time points described below do not include the lead-in treatment period; rather they refer to the length of treatment with the direct antiviral therapy.

“Rapid viral response” or “RVR” in the context of indirect antiviral combination therapy, e.g., comprising a pegylated interferon-alpha and ribavirin, means undetectable serum HCV RNA at the end of four weeks of treatment.

“Early viral response” or “EVR” means a reduction in serum HCV RNA of ≧2 log at the end of 12 weeks of antiviral therapy, with “complete EVR” meaning undetectable serum HCV RNA at the end of 12 weeks of antiviral therapy.

“End of treatment response or “ETR” means undetectable serum HCV RNA at the conclusion of antiviral therapy, and preferably at the conclusion of any of the treatment regimens described herein or at the conclusion of any treatment regimen recommended in prescribing information approved by a regulatory agency. Non-limiting examples of ETR time points are 12, 16, 24, 36 and 48 weeks.

“Sustained viral response” or “SVR” means the undetectable serum HCV RNA at the conclusion of antiviral therapy and at a maximum of 24 weeks following the end of antiviral therapy. In some embodiments, SVR is measured at 12 weeks following the end of antiviral therapy. SVR is also described by Dr. Steven L. Flamm in the Journal of the American Medical Association, Vol, 289, No. 18, pp. 2413 to 2417 (2003).

“Slow response”, in the context of pegylated interferon alpha/ribavirin combination therapy means≧2 log reduction of, but still detectable, serum HCV RNA at the end of 12 weeks of antiviral therapy and undetectable serum HCV RNA at the end of 24 weeks of antiviral therapy.

“Null response” means<1 log reduction in serum HCV RNA and/or <2 log reduction in serum HCV RNA at the end of 4 weeks and 12 weeks of antiviral therapy, respectively.

“Nonresponse” or “NR” means the presence of detectable HCV RNA throughout a minimum of 12 weeks of antiviral therapy. The nonresponse phenotype is typically assigned if serum HCV RNA is detectable at the end of 4 weeks and at the end of 12 weeks of antiviral therapy.

“Relapse” means the presence of detectable HCV RNA at any time after an end of treatment response (ETR), including but not limited to at 12 weeks or 24 weeks after the ETR.

### III. Utility of Ribavirin-Induced Anemia Markers of the Invention

The phenotypic effect of the RIA markers described herein support the use of these markers in a variety of commercial applications, including but not limited to, clinical trials of investigational or previously approved ribavirin drugs in patients selected on the basis of the presence or absence of one or more of these markers, pharmaceutical compositions and drug products comprising a ribavirin compound for treating patients who lack an RIA marker, diagnostic methods, and pharmacogenetic treatment methods, which involve tailoring a patient's drug therapy based on whether the patient has one or more of these markers.

The utility of any of the commercial applications claimed herein does not require that the correlation between the presence of a RIA marker of the invention and the occurrence of hemolytic anemia be observed in 100% of the individuals that receive the ribavirin compound; nor does it require a diagnostic method or kit to have a specific degree of specificity or sensitivity in determining the presence or absence of a RIA marker in every individual, nor does it require that a diagnostic method claimed herein be 100% accurate in predicting for every individual whether the individual is likely to have hemolytic anemia in response to a ribavirin compound. Thus, the inventors herein intend that the terms “determine”, “determining” and “predicting” should not be interpreted as requiring a definite or certain result; instead these terms should be construed as meaning that a claimed method provides an accurate result for the majority of individuals, or that the result or prediction for any given individual is more likely to be correct than incorrect.

Preferably, the accuracy of the result provided by a diagnostic method of the invention is one that a skilled artisan or regulatory authority would consider suitable for the particular application in which the method is used. Similarly, the utility of the claimed drug products and treatment methods does not require that they produce the claimed or desired effect in every individual; all that is required is that a clinical practitioner, when applying his or her professional judgment consistent with all applicable norms, decides that the chance of achieving the claimed effect of treating a given individual according to the claimed method or with the claimed drug product is sufficiently high to warrant prescribing the treatment or drug product.

A. Testing for Ribavirin-Induced Anemia Markers of the Invention

The presence or absence of an RIA marker may be detected by any of a variety of genotyping techniques commonly used in the art. Typically, such genotyping techniques employ one or more oligonucleotides that are complementary to a region containing, or adjacent to, the PS of interest. The sequence of an oligonucleotide used for genotyping a particular PS of interest is typically designed based on a context sequence for the PS. The location, in a particular individual, of any of the polymorphic sites identified in Table 1 is at a position corresponding to the location of the PS of interest in a reference coding or genomic DNA sequence surrounding the PS of interest or in one of the context sequences described in Table 2 below, or their complementary sequences. The context sequences in Table 2 were reported in NCBI SNP Database on Oct. 25, 2009, and the alternative alleles are indicated with the following nomenclature: Y indicates C or T, S indicates G or C, R indicates G or A, K=G or T, M=A or C. Longer context sequences useful in designing oligonucleotides to genotype the PS of Table 1 are the context sequences listed in the NCBI SNP Database as of Oct. 26, 2009.

TABLE 2
Context sequences for SNPs associated with RIA.

| PS | SHORT CONTEXT SEQUENCE | SEQ ID NO |
|---|---|---|
| rs6051702 | AACTCACCATATAACAGGGGTTA | 3 |
| | TTCMTTATATCCTCAAAGAGTGC | |
| | ACTGCC | |
| rs3810560 | TCAGTGGCCCCAAGCCCTCGCTC | 4 |
| | CTCYGGACCCTTGCACATGCTGT | |
| | TCCCAG | |
| rs11697114 | GGGCCCAGGGAGCAGGAAAACA | 5 |
| | CATAYACAAACCCGCCCGCTGAC | |
| | CAGAAAT | |
| rs3310 | CAGAGAGGAACAAAATAAGTTTC | 6 |
| | TGGYTTGGCTGATCTGGGTGATC | |
| | AGGTGG | |
| rs965469 | GGACCAGAAATAAAGCCATACA | 7 |
| | AGTCYAAGTAAGCATACCCTTTT | |
| | TACTTCT | |
| rs6051762 | TAAGCTTGCTGTCCATGATACAG | 8 |
| | TGAYAGAGCAAAACTCCGGTATT | |
| | ATAAAA | |
| rs6051841 | TCACAGCAAAGTTGTAATGGCCT | 9 |
| | CCCRTACTGTCTGTMTCATCATT | |
| | CAGCT | |
| rs6051693 | GTGGCTGTGTGGCTGAAAGACTG | 10 |
| | AATKATAAATTTTGATTTTTATTA | |
| | ATTTA | |
| rs11697114 | GGGCCCAGGGAGCAGGAAAACA | 11 |
| | CATAYACAAACCCGCCCGCTGAC | |
| | CAGAAAT | |
| rs6115892 | CCTGCTCCCTTCCTTCCTATTTTC | 12 |
| | TAYACTTGTCTCACTTCTTGACAT | |
| | GTTC | |
| rs6115865 | GCCTCCTAACAAGATGGAACTAG | 13 |
| | ATGYTGTCAGAGGTAAGAAAGGC | |
| | ACACGC | |
| rs11697620 | CCAGCGTGCGTGTGACACTGTTA | 14 |
| | ACAYGATAGGGGGAGACTGCTTG | |
| | GGGAAA | |
| rs2295547 | TTTCTTTCCTGCCTGTTCGTCCAT | 15 |
| | TAMAAATGCAGGATTCCCAGGGT | |
| | GCCAG | |
| rs8120592 | AGCTACTTTAGCTCCACATAACC | 16 |
| | CAGYTATTTTAGCTCCTTTTCTTG | |
| | AGGTT | |
| rs3827075 | GTTGGGCTCATTGTTGTCAGGGT | 17 |
| | CCCKGGCGAAGCGGCGAAGCAT | |
| | GGTCCGC | |
| rs2326084 | AACCCTTACACCCCAATACCAAC | 18 |
| | ATAMACAGCTATCATTCTCTTCC | |
| | CACTTC | |
| rs1207 | GCCTTTTTGGCTTTGATGCTTCTT | 19 |
| | CAYGTITTGACTMTTCAATCAC | |
| | AGTT | |
| rs10159477 | ATGTTTTCTTTCCCTGGGGAACTC | 20 |
| | ACRCAGTATCATAGGAGATGGAC | |
| | AGCTT | |
| rs6076519 | TAAAATTCAGAGGGAGGAAAGTT | 21 |
| | TTCRTAAGTGAGACACGAAAGGT | |
| | TGAGAT | |
| rs6051689 | TTTCTTTAAGGGCTCCAACTATGC | 22 |
| | CCYGCCATMGTTGCAGGCAGC | |
| | ATAAC | |
| rs1127354 | TCGTTCAGATTCTAGGAGATAAG | 23 |
| | TTTMCATGCACTTTGGTGGCACA | |
| | GAAAAT | |
| rs7270101 | TTGACCGTATGTCTCTGTTTTGTT | 24 |
| | TTMTTTTTAAAAGATGGTTGGAT | |
| | TTCTC | |
| rs7274193 | GGAGCAGTGGTTCACATCTGTAA | 25 |
| | CTCYGGAACTTTGGGAGGCCCAG | |
| | GTGGGA | |
| rs2236094 | CTCTGTGCCTCAGGTATCTAACA | 26 |
| | GATSAAAGGCATGGGTTTAGGAC | |
| | GGCTAA | |
| rs6051708 | GGAAGAGGGGAATCCCGAATGG | 27 |
| | CTGAYTGAACAAGGATGGAAAG | |
| | AAAACCAA | |
| rs6051790 | CCATCTTGCACTGCTGTCGCTTGA | 28 |
| | AGYGGTTTATTAATAATTATTGTT | |
| | TACA | |
| rs6037553 | AAGTTCCTCTAGGTTTTCAAATTT | 29 |
| | TTRTTCTTCTTCCCTGTTAAGATG | |
| | TTCA | |
| rs4611719 | GAAGAATGAAGCCACCGAAGAT | 30 |
| | AGAGRGTTCTGATGACATACTAG | |
| | TGCCTGC | |
| rs2236123 | AAATCTCCTGTGATGCTGGTGTT | 31 |
| | AATSTGCCCTGCATTCTAACCTCA | |
| | GAACA | |
| rs2236118 | ATACTGTAGGCAATTATAACATG | 32 |
| | ATGRTAATATTTGTGAATTTAGG | |
| | CATATC | |
| rs2236122 | CCCCTAGAAACCTCACCATTCAG | 33 |
| | GTAYCCAGCTTTGCCTTTCTAGCC | |
| | TTGGA | |
| rs2236104 | CTGTTTCCAGAGGGAGAAGACCT | 34 |
| | AAAMAAAACAGAATTTGAGCAA | |
| | AGAACAC | |
| rs6037567 | AAAAAAAAAAAAATTTCAACTGA | 35 |
| | TGGMAACTATAAGACAAATGAT | |
| | CTGGTTT | |
| rs6051716 | CGTGTTGCACACTCAAAATAATC | 36 |
| | ATGYTTACAGAGACTTCATGAGC | |
| | TAGTCA | |
| rs6051807 | TTTCCTAGAGCTCGTATTTTCCAT | 37 |
| | ACRTTCAGTTATTACAACATTCA | |
| | CTTGT | |
| rs6051753 | GAAGGAAAAAGGAAGCAATGTG | 38 |
| | TTTCRGTTCAAACATTTCCTAGCT | |
| | GGCTTT | |
| rs6051764 | CCTTCCTCCCTGTCCTCTTGGCTG | 39 |
| | AAYTTTTCCTCTCCCCTACTTTCT | |
| | GCTC | |
| rs1040726 | CGCACTGCCAGGCCCATAGAGAA | 40 |
| | GCAYGCCTGGTAAGCAGGGCTGG | |
| | CGTGTG | |
| rs2281500 | CTCAGAACACACAGGCAAGAGA | 41 |
| | GTGCRTCCGGACCCATCCAGGTC | |
| | AGCAGAC | |
| rs6037554 | TATCCCTTCATCTAGCACAGTGA | 42 |
| | CTGRCATTTACTAGACATTCAAC | |
| | AAGGAT | |
| rs2236089 | TCTGTTTTGGCCTCAAAGGGTTCA | 43 |
| | GAMTAAAAGGGGCTTTTCTCTTG | |
| | TGAGA | |
| rs7270135 | TGTCCCAAATTAGACTAGCAGGA | 44 |
| | ACTYCTTAAGCTACTTCCCATATC | |
| | CTTTT | |
| rs6037560 | TTGTTAAGATGTTTGATTAATGTC | 45 |
| | TTYTTCTCCACTACCCTATAAACT | |
| | TTAT | |
| rs6051713 | AAAACAACAACTAACAGCTCCAC | 46 |
| | TTGRTGTCAAAGTTCAATTCTATT | |
| | GCCCC | |

As recognized by the skilled artisan, nucleic acid samples containing a particular PS may be complementary double stranded molecules and thus reference to a particular site on the sense strand refers as well to the corresponding site on the complementary antisense strand. Similarly, reference to a particular genotype obtained for a PS on both copies of one strand of a chromosome is equivalent to the complementary genotype obtained for the same PS on both copies of the other strand. Thus, an A/A genotype for the rs1127354 PS on the coding strand for the ITPA gene is equivalent to a TIT genotype for that PS on the noncoding strand.

The context sequences recited herein, as well as their complementary sequences, may be used to design probes and primers for genotyping the polymorphic sites of Table 1 in a nucleic acid sample obtained from a human subject of interest using any of a variety of methods well known in the art that permits the determination of whether the individual is heterozygous or homozygous for the anemia allele identified in Table 1. Nucleic acid molecules utilized in such methods generally include RNA, genomic DNA, or cDNA derived from RNA.

Typically, genotyping methods involve assaying a nucleic acid sample prepared from a biological sample obtained from the individual to determine the identity of a nucleotide or nucleotide pair present at one or more polymorphic sites of interest. Nucleic acid samples may be prepared from virtually any biological sample. For example, convenient samples include whole blood serum, semen, saliva, tears, fecal matter, urine, sweat, buccal matter, skin and hair. Somatic cells are preferred since they allow the determination of the identity of both alleles present at the PS of interest.

Nucleic acid samples may be prepared for analysis using any technique known to those skilled in the art. Preferably, such techniques result in the isolation of genomic DNA sufficiently pure for determining the genotype for the desired polymorphic site(s) in the nucleic acid molecule. To enhance the sensitivity and specificity of that determination, it is frequently desirable to amplify from the nucleic acid sample a target region containing the PS to be genotyped. Nucleic acid isolation and amplification techniques may be found, for example, in Sambrook, et al., Molecular Cloning: A Laboratory Manual (Cold Spring Harbor Laboratory, New York) (2001).

Any amplification technique known to those of skill in the art may be used in practicing the present invention including, but not limited to, polymerase chain reaction (PCR) techniques. PCR may be carried out using materials and methods known to those of skill in the art (See generally PCR Technology: Princzals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No. 4,683,202. Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)); isothermal methods (Walker et al., Proc. Natl. Acad. Sci. USA 89:392-6 (1992)); and nucleic acid-based sequence amplification (NASBA).

The amplified target region is assayed to determine the identity of at least one of the alleles present at a PS in the target region. If both alleles of a locus are represented in the amplified target, it will be readily appreciated by the skilled artisan that only one allele will be detected at a PS in individuals who are homozygous at that PS, while two different alleles will be detected if the individual is heterozygous for that PS.

The identity of the allele may be identified directly, known as positive-type identification, or by inference, referred to as negative-type identification. For example, where a SNP is known to be guanine or cytosine in a reference population, a PS may be positively determined to be either guanine or cytosine for an individual homozygous at that site, or both guanine and cytosine, if the individual is heterozygous at that site. Alternatively, the PS may be negatively determined to be not guanine (and thus cytosine/cytosine) or not cytosine (and thus guanine/guanine).

Identifying the allele or pair of alleles (e.g., the two nucleotides in case of a SNP) at a PS in nucleic acid sample obtained from an individual may be accomplished using any technique known to those of skill in the art. Preferred techniques permit rapid, accurate assaying of multiple PS with a minimum of sample handling. Some examples of suitable techniques include, but are not limited to, direct DNA sequencing of the amplified target region, capillary electrophoresis, hybridization of allele-specific probes, single-strand conformation polymorphism analysis, denaturing gradient gel electrophoresis, temperature gradient electrophoresis, mismatch detection; nucleic acid arrays, primer specific extension, protein detection, and other techniques well known in the art. See, for example, Sambrook, et al., Molecular Cloning: A Laboratory Manual (Cold Spring Harbor Laboratory, New York) (2001); Ausubel, et al., Current Protocols in Molecular Biology (John Wiley and Sons, New York) (1997); Orita et al., Proc. Nat. Acad. Sci, USA 86, 2766-2770 (1989); Humphries et al., in MOLECULAR DIAGNOSIS OF GENETIC DISEASES, Elles, ed., pp. 321-340, 1996; Wartell et al., Nucl. Acids Res. 18:2699-706 (1990); Hsu et al. (1994) Carcinogenesis 15:1657-1662; Sheffield et al., Proc. Natl. Acad. Sci. USA 86:232-6 (1989); Winter et al., Proc. Natl. Acad. Sci. USA 82:7575 (1985); Myers et al. (1985) Nature 313:495; Rosenbaum and Reissner (1987) Biophys Chem. 265:12753; Modrich, Ann. Rev. Genet. 25:229-53 (1991); U.S. Pat. No. 6,300,063; U.S. Pat. No. 5,837,832; U.S. Pat. No. 5,459,039; and HuSNP Mapping Assay, reagent kit and user manual, Affymetrix Part No. 90094 (Affymetrix, Santa Clara, Calif.).

In preferred embodiments, the identity of the allele(s) at a PS is determined using a polymerase-mediated primer extension method. Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798.

Another primer extension method employs allele specific PCR (Ruano, G. et al., Nucl. Acids Res. 17:8392 (1989); Ruano, G. et al., Nucl. Acids Res. 19:6877-82 (1991); WO 93/22456; Turki et al., J. Gun. Invest. 95:1635-41 (1995)). In addition, multiple PSs maybe investigated by simultaneously amplifying multiple regions of the nucleic acid using sets of allele-specific primers as described in WO 89/10414.

Yet another primer extension method for identifying and analyzing polymorphisms utilizes single-base extension (SBE) of a fluorescently-labeled primer coupled with fluorescence resonance energy transfer (FRET) between the label of the added base and the label of the primer. Typically, the method, such as that described by Chen et al., Proc. Nat. Acad. Sci. 94:10756-61 (1997) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

A preferred genotyping assay is a TaqMan® SNP Genotyping Assay from Applied Biosystems or an assay having about the same reliability, accuracy and specificity.

In all of the above methods, the accuracy and specificity of an assay designed to detect the identity of the allele(s) at any PS is typically validated by performing the assay on DNA samples in which the identity of the allele(s) at that PS is known. Preferably, a sample representing each possible allele is included in the validation process. For diploid loci such as those on autosomal and X chromosomes, the validation samples will typically include a sample that is homozygous for the major allele at the PS, a sample that is homozygous for the minor allele at the PS, and a sample that is heterozygous at that PS. These validation samples are typically also included as controls when performing the assay on a test sample (i.e., a sample in which the identity of the allele(s) at the PS is unknown). The specificity of an assay may also be confirmed by comparing the assay result for a test sample with the result obtained for the same sample using a different type of assay, such as by determining the sequence of an amplified target region believed to contain the PS of interest and comparing the determined sequence to context sequences accepted in the art, such as the context sequences provided herein.

The length of the context sequence necessary to establish that the correct genomic position is being assayed will vary based on the uniqueness of the sequence in the target region (for example, there may be one or more highly homologous sequences located in other genomic regions). The skilled artisan can readily determine an appropriate length for a context sequence for any PS using known techniques such as blasting the context sequence against publicly available sequence databases. For amplified target regions, which provide a first level of specificity, examining the context sequence of about 30 to 60 bases on each side of the PS in known samples is typically sufficient to ensure that the assay design is specific for the PS of interest. Occasionally, a validated assay may fail to provide an unambiguous result for a test sample. This is usually the result of the sample having DNA of insufficient purity or quantity, and an unambiguous result is usually obtained by repurifying or reisolating the DNA sample or by assaying the sample using a different type of assay.

Further, in performing any of the methods described herein that require determining the presence or absence of a particular RIA marker, or obtaining an individual's genotype for a PS in an RIA marker, such activity may be made by consulting a data repository that contains sufficient information on the patient's genetic composition to determine whether the patient has the marker of interest. Preferably, the data repository lists what RIA marker(s) are present and absent in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a Palm Pilot a tape, a zip disk, the computer's internal ROM (read-only-memory) or the internet or worldwide web. Other media for the storage of files accessible by a computer will be obvious to one skilled in the art.

The invention also contemplates that testing for an RIA marker may be carried out by determining whether the individual has an allele, e.g., nucleotide, at a different locus that is in high linkage disequilibrium (LD) with the anemia allele for any of the SNPs listed in Table 1. Two particular alleles at different loci on the same chromosome are said to be in LD if the presence of one of the alleles at one locus tends to predict the presence of the other allele at the other locus. Such variants, which are referred to herein as linked variants, or proxy variants, may be any type of variant (e.g., a SNP, insertion or deletion) that is in high LD with the anemia allele of interest.

Linked variants are readily identified by determining the degree of linkage disequilibrium (LD) between the anemia allele of any of the SNPs in Table 1 and a candidate linked allele at a polymorphic site located in the chromosomal region 20p13 or elsewhere on chromosome 20. The candidate linked variant may be an allele of a polymorphism that is currently known. Other candidate linked variants may be readily identified by the skilled artisan using any technique well-known in the art for discovering polymorphisms.

The degree of LD between an anemia allele in Table 1 and a candidate linked variant may be determined using any LD measurement known in the art. LD patterns in genomic regions are readily determined empirically in appropriately chosen samples using various techniques known in the art for determining whether any two alleles (e.g., between nucleotides at different PSs) are in linkage disequilibrium (see, e.g., GENETIC DATA ANALYSIS II, Weir, Sineuer Associates, Inc. Publishers, Sunderland, Mass. 1996). The skilled artisan may readily select which method of determining LD will be best suited for a particular population sample size and genomic region. One of the most frequently used measures of linkage disequilibrium is r², which is calculated using the formula described by Devlin et al. (Genomics, 29(2):311-22 (1995)). r² is the measure of how well an allele X at a first locus predicts the occurrence of an allele Y at a second locus on the same chromosome. The measure only reaches 1.0 when the prediction is perfect (e.g. X if and only if Y).

Preferably, the locus of the linked variant is in a genomic region of about 100 kilobases, more preferably about 10 kb that spans any of the PS of Table 1. Other linked variants are those in which the LD with the anemia allele has a r² value, as measured in a suitable reference population, of at least 0.75, more preferably at least 0.80, even more preferably at least 0.85 or at least 0.90, yet more preferably at least 0.95, and most preferably 1.0. The reference population used for this r² measurement may be the general population, a population using the ribavirin compound, a population diagnosed with a particular condition for which the ribavirin compound has activity (such as chronic HCV infection in combination with an interferon alpha) or a population whose members are self-identified as belonging to the same ethnic group, such as Caucasian, African American, Hispanic, Latino, Native American and the like, or any combination of these categories. Preferably the reference population reflects the genetic diversity of the population of patients to be treated with a ribavirin compound.

In some embodiments, a physician determines whether a patient has an RIA marker described herein (or obtains an individual's genotype for a PS in an RIA marker) by ordering a diagnostic test, which is designed to determine whether the patient has at least one anemia allele at one or more of the polymorphic sites in Table 1. Preferably the test determines the identity of both alleles, i.e., the genotype, at this PS. In some embodiments, the testing laboratory will prepare a nucleic acid sample from a biological sample (such as a blood sample or buccal swab) obtained from the patient. In some embodiments, a blood sample from the patient is drawn by the physician or a member of the physician's staff, or by a technician at a diagnostic laboratory. In some embodiments, the patient is provided with a kit for taking a buccal swab from the inside of her cheek, which the patient then gives to the physician's staff member or sends directly to the diagnostic laboratory.

In some embodiments, the testing laboratory does not know the identity of the individual whose sample it is testing; i.e., the sample received by the laboratory is made anonymous in some manner before being sent to the laboratory. For example, the sample may be merely identified by a number or some other code (a “sample ID”) and the results of the diagnostic method can be reported to the party ordering the test using the sample ID. In preferred embodiments, the link between the identity of an individual and the individual's sample is known only to the individual or to the individual's physician.

In some embodiments, after the test results have been obtained, the testing laboratory generates a test report which indicates whether the anemia allele is present or absent at the genotyped polymorphic site, and preferably indicates whether the patient is heterozygous or homozygous for the anemia allele. In some embodiments, the test report is a written document prepared by the testing laboratory and sent to the patient or the patient's physician as a hard copy or via electronic mail. In other embodiments, the test report is generated by a computer program and displayed on a video monitor in the physician's office. The test report may also comprise an oral transmission of the test results directly to the patient or the patient's physician or an authorized employee in the physician's office. Similarly, the test report may comprise a record of the test results that the physician makes in the patient's file.

In one preferred embodiment, if the patient is heterozygous or homozygous for the anemia allele, then the test report further indicates that the patient tested positive for a genetic marker associated with ribavirin-induced anemia, while if the individual is homozygous for the other allele, then the test report further indicates that the patient tested negative for a genetic marker associated with ribavirin-induced anemia. In some embodiments, the test result will include a probability score for having severe anemia in response to the ribavirin compound, which is derived from running a model that weights various patient parameters (e.g., age, gender, ribavirin dose per kilogram, baseline hemoglobin concentration) in the relevant disease population. The weight given to each parameter is based on its contribution relative to the other parameters in explaining the inter-individual variability of anemia exhibited in response to the ribavirin compound in the relevant disease population. The doctor may use this anemia probability score as a guide in selecting a therapy or treatment regimen for the patient. For example, for chronic HCV infection, patient parameters associated with ribavirin-induced anemia include the presence of cirrhosis in addition to age, gender, ribavirin dose per kilogram, and baseline hemoglobin concentration.

Typically, the individual would be tested for the presence of an RIA marker prior to initiation of ribavirin therapy, but it is envisioned that such testing could be performed at any time after the individual is administered the first dose of a ribavirin compound, with preferred testing times being after two weeks, three weeks or four weeks of treatment with the ribavirin compound. For example, the treating physician may be concerned that the patient has not responded adequately and desires to determine whether the individual may be able to tolerate a higher dose of ribavirin by testing for the presence or absence of an RIA marker. In some embodiments, a physician may determine whether or not an individual should be tested for an RIA marker. For example, the physician may be considering whether to prescribe a pharmaceutical composition comprising a ribavirin compound that is indicated for patients who test negative for the RIA marker. In some embodiments, the physician may want to know the patient's RIA marker status to help decide whether to prescribe adjuvant therapy to counteract RBV-induced anemia, such as epoetin alfa.

In deciding how to use the RIA marker test results in treating any individual patient, the physician may also take into account other relevant circumstances, such as the disease or condition to be treated, the age, weight, gender, baseline hemoglobin concentration, genetic background and race of the patient, including inputting a combination of these factors and the genetic marker test results into a model that helps guide the physician in choosing a therapy and/or treatment regimen with that therapy.

Detecting the presence or absence of any of the RIA markers in Table 1 may be performed using a kit that has been specially designed for this purpose. In one embodiment, a kit of the invention comprises a set of oligonucleotides designed for identifying each of the alleles at the PS in at least one marker from Table 1. In preferred embodiments, the PS is rs6051702, rs3810560, rs11697114, rs3310, rs964569, rs1127354 or rs7270101. In another embodiment, the set of oligonucleotides is designed to identify the alleles at any combination of two or more of the PS in Table 1. In a preferred embodiment, the combination of PS comprises at least the rs1127354 and rs7270101 polymorphic sites. In another preferred embodiment, the combination of PS comprises each of rs6051702, rs3810560, rs11697114, rs3310 and rs964569.

In some embodiments, the oligonucleotides in the kit are either allele-specific probes or allele-specific primers. In other embodiments, the kit comprises primer-extension oligonucleotides. In still further embodiments, the set of oligonucleotides is a combination of allele-specific probes, allele-specific primers and primer-extension oligonucleotides. The kit may comprise oligonucleotides designed for detecting the presence of other genetic markers associated with beneficial and/or adverse responses to ribavirin.

Oligonucleotides in kits of the invention must be capable of specifically hybridizing to a target region of a polynucleotide. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. In some embodiments, the target region contains the PS of interest, while in other embodiments, the target region is located one to 10 nucleotides adjacent to the PS.

The composition and length of each oligonucleotide in the kit will depend on the nature of the genomic region containing the PS as well as the type of assay to be performed with the oligonucleotide and is readily determined by the skilled artisan.

For example, the polynucleotide to be used in the assay may constitute an amplification product, and thus the required specificity of the oligonucleotide is with respect to hybridization to the target region in the amplification product rather than in genomic or cDNA isolated from the individual. As another example, if the kit is designed to genotype two or more polymorphic sites simultaneously, the melting temperatures for the oligonucleotides for each PS in the kit will typically be within a narrow range, preferably less than about 5° C. and more preferably less than about 2° C.

In some embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting polymorphisms, departures from complete complementarity are contemplated where such departures do not prevent the molecule from specifically hybridizing to the target region as defined above. For example, an oligonucleotide primer may have a non-complementary fragment at its 5′ end, with the remainder of the primer being completely complementary to the target region. Alternatively, non-complementary nucleotides may be interspersed into the probe or primer as long as the resulting probe or primer is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium.

Typically, stringent conditions include a salt concentration of at least about 0.01 to 1.0 M sodium ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

One non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Stringency conditions with ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tm is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6(log₁₀ [Na+])+0.41(% G+C)−(600/N), where N is the number of bases in the hybrid, and [Na+] is the concentration of sodium ions in the hybridization buffer ([Na+] for 1×SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTEChNOLOGY, A COMPREHENSIVE DESK REFERENCE, Meyers, ed., pp. 617-20, VCH Publishers, Inc., 1995). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

In some embodiments, the set of oligonucleotides in the kit have different labels to allow simultaneous determination of the identity of the alleles at two or more polymorphic sites. The oligonucleotides may also comprise an ordered array of oligonucleotides immobilized on a solid surface such as a microchip, silica beads (such as BeadArray technology from Illumina, San Diego, Calif.), or a glass slide (see, e.g., WO 98/20020 and WO 98/20019). Kits comprising such immobilized oligonucleotides may be designed to perform a variety of polymorphism detection assays, including but not limited to probe hybridization and polymerase extension assays.

Kits of the invention may also contain other reagents such as hybridization buffer (e.g., where the oligonucleotides are to be used as allele-specific probes) or dideoxynucleotide triphosphates (ddNTPs; e.g., where the alleles at the polymorphic sites are to be detected by primer extension). Kits designed for use in polymerase-mediated genotyping assays, may also contain a polymerase and a reaction buffer optimized for the polymerase-mediated assay to be performed.

Kits of the invention may also include reagents to detect when a specific hybridization has occurred or a specific polymerase-mediated extension has occurred. Such detection reagents may include biotin- or fluorescent-tagged oligonucleotides or ddNTPs and/or an enzyme-labeled antibody and one or more substrates that generate a detectable signal when acted on by the enzyme.

It will be understood by the skilled artisan that the set of oligonucleotides and reagents for performing the assay will be provided in separate receptacles placed in the kit container if appropriate to preserve biological or chemical activity and enable proper use in the assay.

In other embodiments, each of the oligonucleotides and all other reagents in the kit have been quality tested for optimal performance in an assay designed to determine the genotype for one or more of the PS in Table 1. In some embodiments, the kit includes an instruction manual that describes how to use the determined genotype to assign, to the tested nucleic acid sample, the presence or absence of an RIA marker.

In some preferred embodiments, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a PS, at a target region containing the PS while not hybridizing to the same region containing a different allele. As understood by the skilled artisan, allele-specificity will depend upon a variety of readily optimized stringency conditions, including salt and formamide concentrations, as well as temperatures for both the hybridization and washing steps.

Examples of hybridization and washing conditions typically used for ASO probes and primers are found in Kogan et al., “Genetic Prediction of Hemophilia A” in PCR PROTOCOLS, A GUIDE TO METHODS AND APPLICATIONS, Academic Press, 1990, and Ruaflo et al., Proc. Natl. Acad. Sci. USA 87:6296-300 (1990).

Typically, an ASO will be perfectly complementary to one allele while containing a single mismatch for the other allele. In ASO probes, the single mismatch is preferably within a central position of the oligonucleotide probe as it aligns with the polymorphic site in the target region (e.g., approximately the 7th or 8th position in a 15 mer, the 8th or 9th position in a 16 mer, and the 10th or 11th position in a 20 mer). The single mismatch in ASO primers is located at the 3′ terminal nucleotide, or preferably at the 3′ penultimate nucleotide. ASO probes and primers hybridizing to either the coding or noncoding strand are contemplated by the invention.

In some embodiments, the kit comprises a pair of allele-specific oligonucleotides for each PS to be assayed, with one member of the pair being specific for one allele (e.g., the anemia allele) and the other member being specific for the other allele. In such embodiments, the oligonucleotides in the pair may have different lengths or have different detectable labels to allow the user of the kit to determine the genotype for the assayed PS.

In still other preferred embodiments, the oligonucleotides in the kit are primer-extension oligonucleotides. Termination mixes for polymerase-mediated extension from any of these oligonucleotides are chosen to terminate extension of the oligonucleotide at the PS of interest, or one base thereafter, depending on the alternative nucleotides present at the PS.

In one embodiment, the kit comprises a pair of allele specific oligonucleotide probes for genotyping at least one of the polymorphic sites in Table 1. In one embodiment, one ASO probe in the pair comprises a nucleotide sequence of at least 15 nucleotides that is identical to or perfectly complementary to the anemia allele of the context sequence shown in Table 2 and the other ASO probe in the pair comprises a nucleotide sequence of at least 15 nucleotides that is identical to or perfectly complementary to the other allele of the context sequence shown in Table 2. In one preferred embodiment, the kit comprises such ASO probes for genotyping at least one PS selected from the group consisting of rs6051702, rs3810560, rs11697114, rs3310, rs964569, rs1127354 and rs7270101. In another preferred embodiment, the kit comprises such ASO probes for genotyping any two or more of these PS such as (a) rs1127354 and rs7270101 or (b) rs6051702, rs3810560 and rs11697114. In still another embodiment, the kit comprises such ASO probes for genotyping each of rs6051702, rs3810560, rs11697114, rs3310, rs964569, rs1127354 and rs7270101.

In yet another embodiment, the susceptibility of an individual for ribavirin-induced anemia is predicted by determining the individual's phenotype for erythrocyte ITPA activity. This phenotyping method will be useful to detect individuals at low risk for ribavirin-induced anemia because they have ITPA deficiency caused by a polymorphism or mutation other than rs1127354 and rs7270101, or caused by other factors that influence the level of ITPA activity (e.g., cigarette smoke, diet, steroid oral contraceptives and other drugs, Atanasova S. et al., Ther Drug Monit 29(1):6-10 (2007)). The ITPA activity is measured in an erythrocyte lysate prepared from a blood sample obtained from the individual, Assays for measuring ITPA activity in human erythrocytes have been described, see, e.g., Holmes S. L., et al., Clin Chim Acta 97(2-3):143-153 (1979); Sumi S., et al., Hum Genet, 111:36-370 (2002); Bireau, J., et al., Nucleosides Nucleotide Nucleic Acids 25(9-11):1129-1132 (2006); and Shipkova et al., Clin. Chem. 52(2):240-247 (2006). Also, the ITPA activity levels for various genotypes at rs1127354 and rs7270101 have been reported: Sumi et al., supra and Shipkova et al., supra. Table 3 below presents the median and range of ITPase activity for different genotypes of the rs1127354 and rs7270101 polymorphic sites that would be expected using the assay and data described in Shipkova et al., supra.

TABLE 3
ITPA genotype-phenotype correlation in healthy Caucasians^{a}.

| | ITPA activity |
|---|---|
| | μmol IMP/(g Hb · h) |
| ITPA Genotype | Median (range) |
| rs1127354/rs7270101: CC/AA | 254.6 (133.9-408.3) |
| rs1127354: AA | 0.3 |
| rs1127354: CA | 65.0 (40.0-104.1) |
| rs7270101: CC | 75.8 (70.7-88.3) |
| rs7270101: AC | 155.2 (95.5-277.6) |
| rs1127354/rs7270101: CA/AC | 20.8 (12.4-24.3) |

^{a}Extracted from Table 2 of Shipkova et al., supra.

In preferred embodiments, an individual with an erythrocyte ITPA activity of ≧125 mmol IMP/g Hb×h (as determined by the assay described in Shipkova et al., supra) would be expected to exhibit a degree of ribavirin-induced anemia that is comparable to that exhibited by patients who have a heterozygous RIA marker (CA genotype) for rs1127354.

B. Pharmaceutical Compositions, Drug Products and Treatment Regimens

An individual to be tested in, or treated by, any of the methods and products described herein is a human subject in need of treatment with a ribavirin compound. In some embodiments, the individual has been diagnosed with, or exhibits a symptom of, a disease susceptible to treatment with the ribavirin compound. In other embodiments, the ribavirin compound to be used has been approved for use in treating an indication with which the individual has been diagnosed. In yet other embodiments, the ribavirin compound to be used is not approved for treating the diagnosed disease or exhibited symptom(s), but the prescribing physician believes the drug may be helpful in treating the individual.

The ribavirin compound used in the pharmaceutical compositions, drug products and methods of the present invention may be any nucleoside analogue, including any ribavirin derivative, which is metabolized in erythrocytes to generate a triphosphate that is structurally similar to RTP. Thus, ribavirin compounds useful in the present invention include, but are not limited to, ribavirin prodrugs that metabolize in vivo into ribavirin. Such ribavirin prodrugs include the ribavirin derivatives described in U.S. Pat. No. 6,673,773, with a preferred ribavirin prodrug having the formula I:

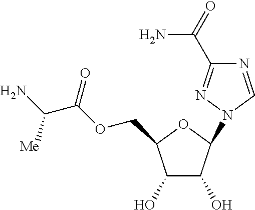

Another preferred ribavirin prodrug is taribavirin (1-(β-D-Ribofuranosyl)-1,2,4-triazole-3-carboximide, also known as viramidine and ribamidine). Pro-drugs of taribavirin are also useful as ribavirin compounds in the present invention, including the viramidine prodrugs described in WO 01/60379.

The ribavirin compound may be formulated for oral, intravenous or airway administration. Preferred formulations of ribavirin include a capsule marketed as REBETOL® by Schering-Plough, a tablet marketed as COPEGUS by Hoffmann La-Roche, a solution for inhalation marketed as VIRAZOLE® by Valeant Pharmaceuticals, and generic versions of the aforementioned branded products, including RIBASPHERE® tablets marketed by Three Rivers Pharmaceuticals, capsules and tablets marketed by Teva Pharmaceuticals Industries Ltd and ribavirin capsules and tablets marketed by Sandoz.

Diseases and conditions that may be treated in accordance with the present invention are generally those that are susceptible to treatment with a ribavirin compound, i.e., the ribavirin compound achieves a clinically measurable beneficial result in a group of patients with the disease, e.g., reduction in viral load in HCV-infected patients. Exemplary diseases and conditions susceptible to treatment with a ribavirin compound include but are not limited to viral infections caused by a wide range of RNA and DNA viruses, including, but not limited to, hepatitis A virus, hepatitis B virus, hepatitis C virus, hepatitis D virus, yellow fever virus, Dengue virus, West Nile virus, Kunjin virus, influenza A, B and C viruses (including H1N1 and other swine influenza viruses), human parainfluenza viruses, respiratory syncytial virus (“RSV”); SARS coronavirus, measles virus, smallpox virus, Lassa fever virus; Korean Haemorrhagic fever virus, Crimean-Congo Haemorrhagic virus, human immunodeficiency virus (HIV), St. Louis encephalitis virus, hantavirus, polio, Canine distemper virus, adenovirus, herpes virus, human herpes virus type 6, papilloma virus, poxvirus, rhinovirus, human T lymphotropic virus-type 1 and 2, human rotavirus and rabies virus. Preferably, the disease is one for which the ribavirin compound has been approved by a regulatory agency such as the U.S. Food and Drug Administration.

In preferred embodiments, the viral infection is HCV and the ribavirin compound is used in combination with at least one other antiviral agent such as an interferon, including an interferon alpha (IFN-α), an interferon lambda (e.g., IFN-λ1, IFN-λ2 or IFN-λ3) and interferon beta (IFN-β). In a particularly preferred embodiment, the viral infection is chronic HCV infection and the at least one other antiviral agent is recombinant IFN-α2a or IFN-α2b or any consensus IFN-α protein in which the amino acid sequence has been designed by selecting at each position the amino acid that most commonly occurs at that position in the various native IFN-α subtypes.

Particularly preferred IFN-α compositions for use in combination with a ribavrin compound in the methods of the present invention are interferon alpha-2 products approved by a government regulatory agency, including any of the following: Roferon®-A (Interferon-alfa 2A, recombinant) marketed by Hoffmann La-Roche, Nutley N.J.), and pegylated versions thereof, such as PEGASYS® (peginterferon alfa-2a) marketed by Hoffmann La-Roche, Nutley N.J.); INTRON® A (Interferon alfa-2b, recombinant) marketed by Schering Corporation, Kenilworth, N.J.) and pegylated versions thereof, such as PegIntron® (peginterferon alfa-2b); (INFERGEN®(Interferon alfacon-1), a consensus IFN-α originally developed by Amgen, Thousand Oaks, Calif. and currently marketed by Three Rivers Pharmaceuticals, Warrendale, Pa. Other interferons contemplated for use in the present invention include: fusions between interferon alpha and a non-interferon protein, such as ZALBIN® (albinterferon alfa-2b), which is being developed by Human Genome Sciences, Rockville, Md. and Norvartis, Basel, Switzerland; Locteron, an investigational controlled release interferon alpha formulation (Biolex/OctoPlus); and Belerofon®, a single amino acid variant of natural alpha interferon, engineered by Nautilus Biotech. Any of the above-named IFN-α compositions may also be sold under different trade names, such as VIRAFERONPEG® peginterferon alfa-2b, which is the same composition as PegIntron® peginterferon alfa-2b.

PEGASYS® peginterferon alfa-2a is obtained by covalent binding of one 40 kDa branched PEG-polymer via an amide bond to a lysine side chain of an interferon alpha-2b molecule, see, e.g., Dhalluin, C. et al., Bioconjugate Chem. 16:504-517 (2005) and U.S. Pat. No. 7,201,897. The resulting product is a mixture of mainly six monopegylated positional isomers (Dhalluin, C., supra, Poser, S. et al., J. Prot. Exp. Purif. 30: 78-87 [2003]). PEGASYS® (peginterferon alfa-2a) and biosimilars thereof are also referred to herein as bPEG40K-interferon alfa-2a.

PegIntron® peginterferon alfa-2b is obtained by covalently reacting recombinant interferon-alfa 2b with a succinimidylcarbonate PEG having an average molecular weight of 12,000 Da (SC-PEG12k) in 100 mM sodium phosphate, pH 6.5 (see, e.g., Grace, M. et al., J. Interferon Cytokine Res. 21:1103-4115 (2001); Wang, Y. S. et al., Adv. Drug Delivery Rev. 54:547-570 (2000); and U.S. Pat. No. 5,951,974). The resulting product is a mixture of mainly monopegylated species in which the PEG12k is attached to different residues of interferon alfa-2b via a urethane bond, with the majority positional isomer having the urethane bond at Histidine 34 (see, e.g., Wang, Y. S. et al., supra and U.S. Pat. No. 5,951,974). PegIntron® peginterferon alfa-2b and biosimilars thereof are also referred to herein as PEG12k-interferon alfa-2b.

Other previously approved and currently marketed IFN-α products that may be used in the methods of the present invention include: Berofor® alpha 2 (recombinant interferon alpha-2C, Boehringer Ingelheim Pharmaceutical, Inc., Ridgefield, Conn.; interferon alpha-n1, a purified blend of natural alpha interferons known as Surniferon® (Sumitomo, Japan) or as Wellferon® interferon alpha-n1 (INS), Glaxo-Wellcome Ltd., London, Great Britain; a consensus alpha interferon such as those described in U.S. Pat. Nos. 4,897,471 and 4,695,623 (especially Examples 7, 8 or 9 thereof); ALFERON N Injection® [Interferon alfa-n3 (human leukocyte derived), a mixture of multiple species of natural alpha interferons available from Hemispherx Biopharma, Inc., Philadelphia, Pa.

Other interferon alpha-polymer conjugates useful in the present invention are described in U.S. Pat. No. 4,766,106, U.S. Pat. No. 4,917,888, European Patent Application No. 0 236 987, European Patent Application Nos. 0 510 356, 0 593 868 and 0 809 996 and International Publication No. WO 95/13090.

Also contemplated for use in the present invention is any pegylated interferon alpha 2a or 2b pharmaceutical composition that is approved by a regulatory agency based, at least in part, by reliance on the preclinical and/or clinical data previously submitted to the regulatory authority in connection with approval of any of the above-described marketed pegylated interferon alpha products, i.e., PEGASYS® (peginterferon alfa-2a) and PegIntron® (peginterferon alfa-2b). Such later approved products may be described by the regulatory agency in terms such as a generic of, bioequivalent to, a biosimilar of, or a substitute for the previously approved product, which terms may or may not be explicitly defined by the regulatory agency.

Pharmaceutical compositions of pegylated interferon alphas intended for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose, trehalose), carriers (e.g. human serum albumin), toxicity agents (e.g. NaCl), preservatives (e.g. thimerosol, cresol or benzylalcohol), and surfactants (e.g. tween or polysorbates) in sterile water for injection. See, e.g., U.S. Pat. No. 6,180,096 and International Patent Application WO2006/020720. Such compositions may be stored as lyophilized powders under refrigeration at 2°-8° C. and reconstituted with sterile water prior to use. Such reconstituted aqueous solutions are typically stable when stored between and used within 24 hours of reconstitution. See, for example, U.S. Pat. Nos. 4,492,537; 5,762,923 and 5,766,582. Lyophilized pegylated interferon formulations may be provided in a pen-type syringe system that comprises a glass cartridge containing a diluent (i.e., sterile water) in one compartment and the lyophilized pegylated interferon-alpha powder in a separate compartment.

Examples of aqueous pegylated interferon formulations are described in U.S. Pat. No. 5,762,923. Such formulations may be stored in prefilled, multi-dose syringes such as those useful for delivery of drugs such as insulin. Typical suitable syringes include systems comprising a pre-filled vial attached to a pen-type syringe such as the NOVOLET Novo Pen available from Novo Nordisk, as well as prefilled, pen-type syringes which allow easy self-injection by the user.

The present invention also contemplates the use of a ribavirin compound and any of the above Interferon alphas in combination with a toll like receptor (TLR) agonist, which are proposed to induce interferon response. For example, agonists for TLR3, TLR7 and TLR9 are being evaluated for use in treating HCV.

In preferred embodiments, the RIA markers of the present invention are used in conjunction with a interferon alpha/ribavirin combination therapy treatment regimen approved by a regulatory authority for a chronic HBV or chronic HCV indication, and in particularly preferred embodiments, in conjunction with any of the dosing and treatment regimens for chronic hepatitis C described in the Package Inserts for the Roferon®-A (Interferon-alfa 2A, recombinant), PEGASYS® (peginterferon alfa-2a), INTRON® A (Interferon alfa-2b, recombinant) and PegIntron® (peginterferon alfa-2b) products. For the PegIntron® (peginterferon alfa-2b) product, such approved combination regimens recommend therapy for 24 weeks for patients chronically infected with HCV genotype 2 or 3, and up to 48 weeks for patients chronically infected with HCV genotype 1, with 24 weeks therapy approved in Europe for the subset of patients with genotype 1 infection and low viral load (<600,000) patients who are HCV-RNA negative at treatment week four and remain HCV-RNA negative at treatment week 24.

The invention also contemplates the use of a nucleoside analog with less anemia potential than ribavirin in combination with an IFN-α-based regimen for treating HCV infection in individuals who test positive for an RIA marker. For example, patients treated with taribavirin and PegIntron (peginterferon alfa-2b) in clinical trials reportedly exhibited less anemia than patients treated with ribavirin and PegIntron.

The RIA markers of the present invention may also be used to select patients chronically infected with HCV who are least likely to develop RBV-induced anemia upon treatment with IFN-α/ribavirin therapy in combination with one or more additional antiviral agents. Alternatively, patients who test positive for an RIA marker might be prescribed one or more antiviral agents that are not a ribavirin compound with or without an IFN-α. Non-limiting examples of antiviral agents useful in such combination treatment regimens include an HCV protease inhibitor, an NS3 protease inhibitor, an HCV polymerase inhibitor, an HCV NSSA inhibitor, an IRES inhibitor, an NS4B inhibitor, an HCV helicase inhibitor, an HCV entry inhibitor, an HCV virion production inhibitor, and other interferons.

In one embodiment, the antiviral agent is an HCV protease inhibitor.

HCV protease inhibitors useful in such combination regimens are described in published international application nos. WO2009/038663, WO 2007/092616, and WO 2002/18369 and in published U.S. Patent Application 2007/0042968.

Other HCV protease inhibitors useful in the methods and combination therapies of the present invention include boceprevir (SCH503034) and SCH 900518 (Schering-Plough); telaprevir (VX-950), VX-500 and VX-813 (Vertex Pharmaceuticals); MK-7009 (Merck); and ITMN-191 (R7227) (Intermune and Roche); TMC-435 (Medivir/Tibotec); MK-7009 (Merck); GS-9132 and ACH-1095 (Gilead/Achillon); PHX1766 (Phenomix); ABT-450 HCV (Abbott/Enanta Pharmaceuticals); and BILN 2061 and BI 201335 (Boehringer Ingelheim).

Additional examples of HCV protease inhibitors useful in the methods and combination therapies of the present invention include those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); Bio World Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, the following compounds:

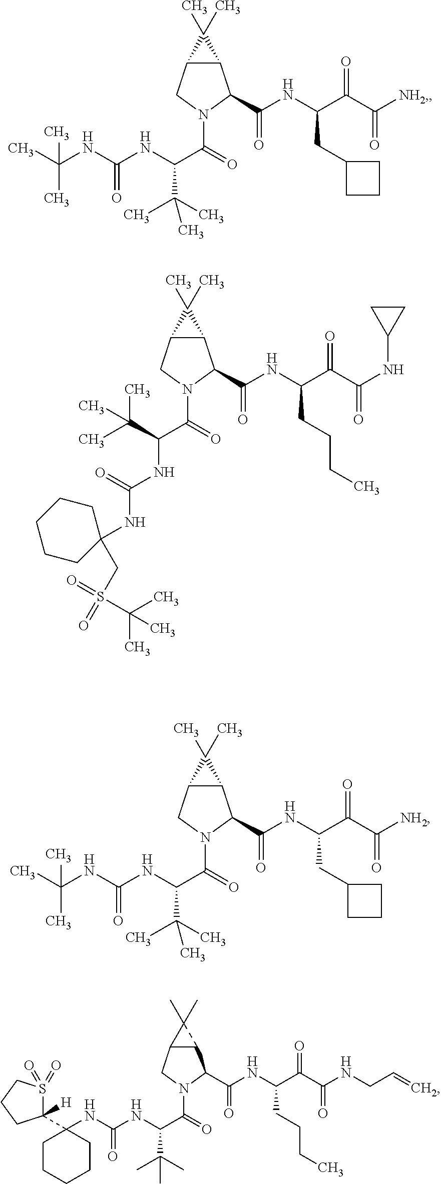

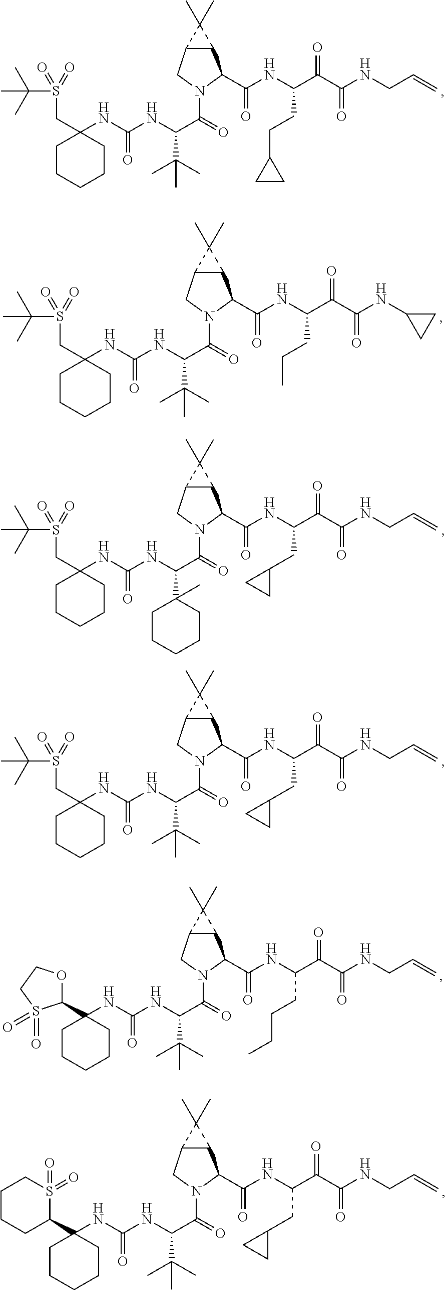

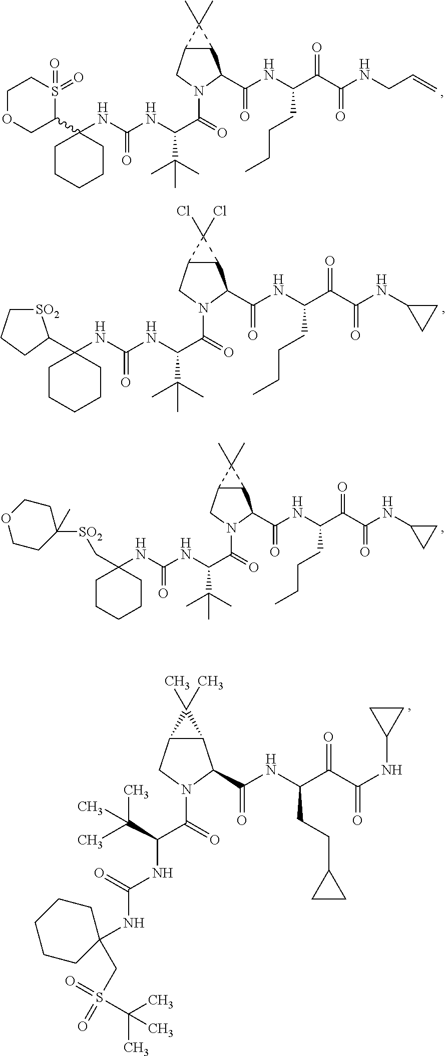

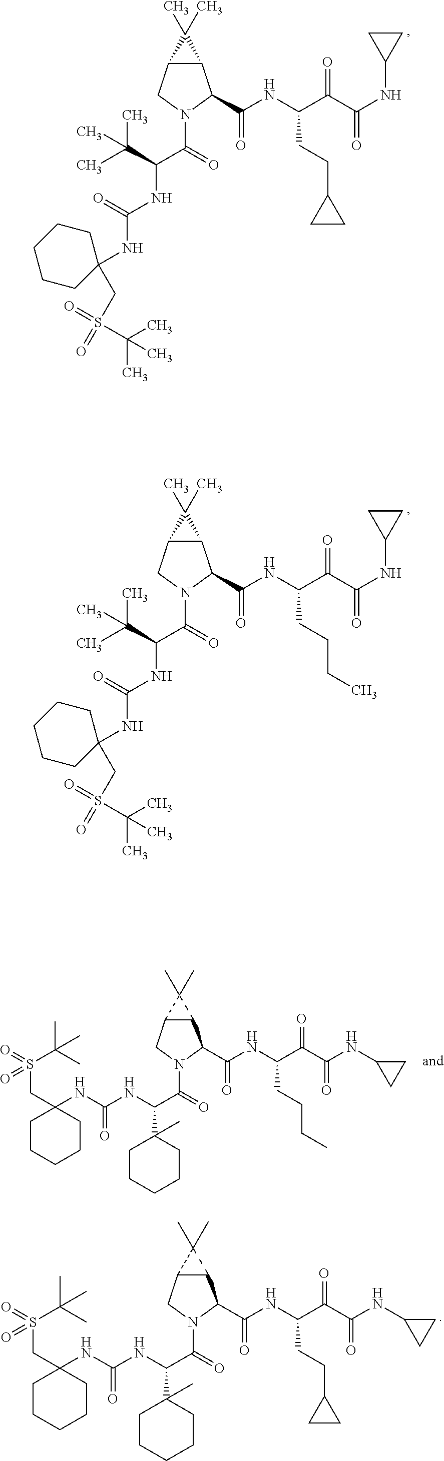

In another embodiment, the antiviral agent is an NS3 protease inhibitor. NS3 serine protease inhibitors useful in the present methods and combination therapies of the present invention include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124,148.

In a still further embodiment, the antiviral agent is an HCV polymerase inhibitor. HCV polymerase inhibitors useful in the methods and combination therapies of the present invention include, but are not limited to: VP-19744 (Wyeth/ViroPharma), PSI-7851 (Pharmasset), R7128 (Roche/Pharmasset), PF-00868554 (Pfizer), VCH-759 and VCH-916 (ViroChem/Vertex), HCV-796 (Wyeth/ViroPharma), IDX184 (Idenix), NM-283 (Idenix/Novartis), R-1626 (Roche), MK-0608 (Isis/Merck), GS 9190 (Gilead), ABT-333 (Abbott), A-848837 and A-837093 (Abbott), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004), and International Publication Nos. WO 08/082,484, WO 08/082,488, WO 08/083,351, WO 08/136,815, WO 09/032,116, WO 09/032,123, WO 09/032,124 and WO 09/032,125.

In another embodiment, the antiviral agent is an HCV NS5A inhibitor. Nonlimiting examples of HCV NS5A inhibitors useful in the methods and combination therapies of the present invention are AZD2836 (A-831) and AZD7295 (A-689) (Arrow Therapeutics); and BMS-790052 (Bristol-Myers Squibb).

In one embodiment the antiviral agent is an NS4B inhibitor, such as clemizole hydrochloride and other salts of clemizole.

In one embodiment, the antiviral agent is a HCV replicase inhibitor including those disclosed in U.S. Patent Publication No. US20090081636.

In another embodiment, the antiviral agent is an HCV helicase inhibitor such as trioxsalen.

In another embodiment, the antiviral agent is an HCV entry inhibitor, including but not limited to ITX5061 and ITX4520 (iTherx)), PRO206 (Progenies) and celgosivir (MX-3253), MIGENIX.

In another embodiment the antiviral agent is an RNAi compound, e.g., TT-033 (Tacere Therapeutics, Inc., San Jose, Calif.).

In a still further embodiment, the antiviral agent is another Type 1 interferon (e.g., IFN-beta or IFN-omega), a Type II interferon (e.g., IFN-gamma or a Type III interferon (e.g., Il-28 or Il-29).

Examples of Type III interferons contemplated for use in the methods and combination therapies of the present invention include, but are not limited to PEG-IFN lambda (ZymoGenetics/Brisol Myers Squibb).

Examples of further additional antiviral agents contemplated for use in the methods and combination therapies of the present invention include, but are not limited to, TT033 (Benitec/Tacere Bio/Pfizer), Sirna-034 (Sirna Therapeutics), GNI-104 (GENimmune), IDX-102 (Idenix), Levovirin™ (ICN Pharmaceuticals, Costa Mesa, Calif.); Humax (Genmab), ITX-2155 (Ithrex/Novartis), PRO206 (Progenies), HepaCide-I (NanoVirocides), MX3235 (Migenix), SCV-07 (SciClone Pharma), KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pennsylvania); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128, ITMN-191 and ribavirin); and mycophenolate mofetil (Hoffman-LaRoche, Nutley, N.J.), SCY-635 (SCYNEXIS), ANA773 (Anadys), CYT107 (Cytheris), SPC3649 (Santaris Pharma), Alinia (nitrazoxanide) (Romark); Oglufanide disodium (Implicit Bioscience), CTS-1027 (Conatus) NOV-205 (Novelos Therapeutics), IMO-2125 (Idera Pharmaceuticals) and CF102 (CAN-FITE).

For individuals who have one or both of (1) a positive test for the presence of an RIA marker and (2) a negative test for ITPA deficiency (e.g. ITPA activity≧125 μmol IMP/g Hb×h, as determined by the assay described in Shipkova et al., supra), the invention also contemplates adjuvant therapy with an agent that counteracts RBV-induced anemia to any therapeutic-regimen that contains a ribavirin compound. Such agents include epoieten alfa, Kampo medicine juzen-taiho-to (TJ-48), ninhinyoeito (NYT) and eicosapentaenoic acid (EPA) with or without vitamins C and E supplementation, see, e.g., Martin, P., et al., J Gastroenterol and Hepatol 23:844-855 (2008), or an agent that inhibits erythrocyte ITPA activity.

In some embodiments, patients with a disease susceptible to treatment with ribavirin, but who test positive for an RIA marker and/or test negative for ITPA deficiency (e.g., ITPA activity≧125 μmol IMP/g Hb×h) are treated with a treatment regimen that excludes a ribavirin compound. In some embodiments, such treatment regimens comprise an inhibitor of inosine monophosphate dehydrogenase (IMPDH) that is not a ribavirin compound, such as merimepodib (VX-497) (Markland W., et al., Antimicrob Agents Chemother 44:859-866 (2000)), mycophenolate mofetil (Kornberg A. et al., Int. Immunopharmacol. 5:107-115 (2005)) and mizoribine (Naka K. et al., Biochem. Biophys. Res Commun. 330:871-879 (2005).

The doses and dosage regimen of the other agents used in the combination therapies of the present invention for the treatment of an HCV infection can be determined by the attending clinician, taking into consideration the approved doses and dosage regimen in the package insert; and the age, sex and general health of the patient. Agents administered in HCV combination therapy can be administered simultaneously (i.e., in the same composition or in separate compositions one right after the other) or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

When the IFN-α is a PEG12k-interferon alfa-2b such as PegIntron® (peginterferon alfa-2b) or a biosimilar thereof, a preferred treatment regimen for chronic HCV infection comprises 1.5 mcg/kg of the PEG12k-interferon alfa-2b once a week in combination with daily doses of 800-1400 mg ribavirin. The ribavirin dose is based on patient weight: 800 mg/day for patients weighing 40-65 kg, 1000 mg/day for patients weighing more than 65 and up to 85 kg, 1200 mg/day for patients weighing more than 85 and up to 105 kg, and 1400 mg/day for patients weighing more than 105 kg. In some embodiments, the recommended weekly dose of the PEG12k-interferon alfa-2b is 0.5, 0.75 or 1.0 mcg/kg and the daily ribavirin dose is between 600-1400 mg ribavirin, based on patient weight.

When IFN-α is a bPEG40K-interferon alfa-2a such as PEGASYS® (peginterferon alfa-2a) or a biosimilar thereof, a preferred treatment regimen for chronic HCV infection comprises 180 mcg/week of the bPEG40K-interferon alfa-2a in combination with a daily ribavirin dose of 1000 mg for patients weighing <75 kg and 1200 mg for patients weighing ≧75 kg. In some embodiments, the recommended weekly dose of the bPEG40K-interferon alfa-2a is at least 25% less than 180 mcg.

In some preferred embodiments, patients who are chronically infected with high viral load HCV genotype 1 and test negative for an RIA marker are treated with a combination regimen that comprises a lead-in treatment period of about 2 to 17 weeks, in which an interferon alpha such as a PEG12k-interferon alfa-2b and a bPEG40K-interferon alfa-2a is administered in combination with ribavirin or another ribavirin compound, followed by a second treatment period of about 12 to about 28 weeks in which a triple combination of the interferon alpha, ribavirin compound and a protease inhibitor such as boceprevir or telaprevir is administered. Such two phase treatment regimens are described in the international patent application publication WO 2009/038663. In particularly preferred embodiments, the lead-in period is about 4 weeks and the second treatment period is about 24 weeks.

When administering a combination therapy that is selected to treat a patient based on the presence or absence in the patient of an RIA genetic marker or ITPA deficiency biomarker, the therapeutic agents in the combination, or a pharmaceutical composition or compositions comprising the therapeutic agents, may be administered in any order such as, for example, sequentially, concurrently, together, simultaneously and the like. The amounts of the various therapeutic agents in such combination therapy may be different amounts (different dosage amounts) or same amounts (same dosage amounts). In some embodiments, the agents in the combination are administered in doses commonly employed when such agents are used as monotherapy for treating the patient's disease or condition, while in other embodiments, the agents are administered in doses lower than the doses commonly employed when such agents are used as monotherapy for treating the disease or condition.

In some embodiments, the therapeutic agents used in combination therapy are present in the same pharmaceutical composition, which may be suitable for oral administration, intravenous administration, subcutaneous administration or parenteral administration.

The inventors herein also contemplate that the RIA markers described herein could be used to seek regulatory approval to market a new ribavirin drug product for a pharmacogenetic indication, i.e., an indication that includes a disease component and an RIA marker component. The disease component is a disease susceptible to treatment with a ribavirin compound and the genetic marker component is a patient who tests negative for at least one of the RIA markers described herein. Similarly, the inventors herein contemplate that these RIA markers are useful for seeking approval of such pharmacogenetic indications for currently approved ribavirin drugs that physicians are reluctant to prescribe for certain diseases based on the marginal benefit/risk ratio of the drug for such diseases in the general population.

Seeking approval for a pharmacogenetic indication typically involves measuring the incidence of anemia in response to a ribavirin compound in two separate groups of patients treated with the compound. Each individual within one of the groups has disease and genetic profiles that place the individual within the proposed pharmacogenetic indication. The individuals in the other group may be randomly selected without regard to whether they have the genetic marker component of the proposed pharmacogenetic indication. Alternately, the individuals are assigned to the other group in a manner that results in a “control” group in which the percentage of individuals who meet and do not meet the genetic marker component is similar to what is observed in the general population, or in a population of patients with the disease component of the proposed pharmacogenetic indication. The drug product for which approval is sought could be administered to the two groups in a prospective trial. Alternatively, a retrospective pharmacogenetic analysis of patients previously treated with the drug could be performed.

The drug product for which a pharmacogenetic indication is being sought could be evaluated with other therapeutically active agents, for example another drug with efficacy for treating the disease or condition in the proposed pharmacogenetic indication or an agent that is intended to reduce the incidence of an adverse effect other than anemia that is caused by ribavirin. In some embodiments, the pharmacogenetic indication for which regulatory approval is sought may include other markers (genetic markers or biomarkers) or predictors of response to the drug. For example, genetic markers that are associated with SVR in chronic HCV patients treated with combination PegIFN/ribavirin therapy are described in Ge D, Fellay J, Thompson A J, et al. Genetic variation in IL2813 predicts hepatitis C treatment-induced viral clearance. Nature 2009 and in US provisional application 61232547 filed 14 Aug. 2009. Also, rapid HCV viral response (RVR) to combination therapy with pegylated interferon alpha and ribavirin is a good predictor of achieving SVR.

The pharmacogenetic study could be designed in consultation with representatives of the regulatory agency or government entity from whom approval is required before marketing the pharmacogenetic drug product in a particular country, Preferably, the regulatory agency is authorized by the government of a major industrialized country, such as Australia, Canada, China, a member of the European Union, Japan, and the like. Most preferably the regulatory agency is authorized by the government of the United States and the type of application for approval that is filed will depend on the legal requirements set forth in the last enacted version of the Food, Drug and Cosmetic Act that are applicable for the drug product and may also include other considerations such as the cost of making the regulatory filing and the marketing strategy for the drug product. For example, if the pharmaceutical formulation in the drug product has previously been approved for the disease component of the proposed pharmacogenetic indication, then the application might be a paper NDA, a supplemental NDA or an abbreviated NDA, but the application would might need to be a full NDA if the pharmaceutical formulation has never been approved before; with these terms having the meanings applied to them by those skilled in the pharmaceutical arts or as defined in the Drug Price Competition and Patent Term Restoration Act of 1984.

One desired outcome of a pharmacogenetic clinical trial using one or more of the RIA markers of the invention is approval to market a drug product which comprises (1) a pharmaceutical composition comprising a ribavirin compound and (2) prescribing information which includes a pharmacogenetic indication for which the pharmaceutical composition is recommended. Prescribing information is typically found in the product insert, also frequently referred to as the package insert or label, for the drug.

As discussed above, the pharmacogenetic indication has two components: a disease component and RIA marker component. Thus, the prescribing information would describe a genetically defined group of patients for which the drug has demonstrated less anemia in the treatment of the disease or diseases listed in the disease component. In some embodiments, the prescribing information will discuss how to identify individuals who are in the genetically defined group. For example, in some embodiments, the prescribing information states that the drug is indicated for individuals who test negative for one or more of the RIA markers described herein or who test positive for ITPA deficiency. Alternately, the prescribing information may state that the drug is contraindicated for individuals who test positive for one or more of the RIA markers or who test negative for ITPA deficiency. In some preferred embodiments, the prescribing information includes the name of at least one approved diagnostic test to be used for detecting the presence or absence of the required genetic marker component of the pharmacogenetic indication. As described above, the pharmacogenetic indication in a pharmacogenetic drug product of the invention may include additional markers or predictors of response to the pharmaceutical composition and/or a requirement to use the drug in combination with one or more other therapeutically active agents. The prescribing information may include information on recommended dosages and treatment regimens.

In some embodiments, the pharmacogenetic drug product is provided as a formulation or in packaging that has a distinctive appearance that the manufacturer has adopted to identify the drug product as a pharmacogenetic product to aid pharmacists and physicians in distinguishing this product from other marketed products comprising the same or similar active ingredient, but which do not have a pharmacogenetic indication. Using the appearance of pharmaceutical formulations and drug product packaging as part of creating a distinctive brand for drug products is well known in the art, and includes the shape and color of tablets or capsules, as well as symbols or logos stamped thereon, or on the packaging material for the drug product.

In preferred pharmacogenetic drug products of the invention, the pharmaceutical composition comprises ribavirin. A preferred pharmacogenetic indication for drug products of the invention comprises the use of the pharmaceutical composition in combination with an interferon alpha for the treatment of patients chronically infected with HCV and who test negative for at least one of the RIA genetic markers described in Table 1. In some preferred embodiments, the patients have a high baseline HCV viral load, as defined hereinabove. In other preferred embodiments, the patients are infected with HCV genotype 1 and have a high HCV viral load. In more preferred embodiments, the prescribing information states that the ribavirin pharmaceutical composition is indicated in combination with an interferon alpha and at least one other antiviral agent for treating patients chronically infected with a high baseline viral load of HCV genotype 1. The antiviral agent may be an HCV protease inhibitor, HCV polymerase inhibitor or another agent that specifically inhibits HCV replication. The prescribing information may recommend the use of the ribavirin pharmaceutical composition in combination with any combination of two or more of these antiviral agents. In addition, the prescribing information may include a recommended treatment regimen, with preferred treatment regimens being any of those described above for PEG12k-interferon alfa-2b and bPEG40K-interferon alfa-2a pharmaceutical compositions.

Any or all analytical and mathematical operations involved in performing the methods described herein or in using the kits and products described herein may be implemented by a computer. For example, the computer may execute a computer program that assigns the presence or absence of an RIA marker to an individual based on genotype data inputted by an employee of a testing laboratory or by the treating physician. In addition, the same computer or a different computer may output a degree of anemia that is predicted to occur in the individual based on the RIA marker assignment and optionally other patient-specific or therapy-specific factors that may affect RBV-induced anemia. In some embodiments, the computer executes a computer program that derives an anemia probability score for the patient from various patient and disease parameters associated with RBV-induced anemia, such as the presence or absence of one or more RIA markers, ITPA activity level, baseline hemoglobin level, concomitant medicines, etc. Data relating to the presence or absence of RIA markers or ITPA deficiency in an individual may be stored as part of a relational database (e.g., an instance of an Oracle database or a set of ASCII flat files) containing other clinical and/or genetic data for the individual. These data may be stored on the computer's hard drive or may, for example, be stored on a CD ROM or on one or more other storage devices accessible by the computer. For example, the data may be stored on one or more databases in communication with the computer via a network.

### IV. Exemplary Specific Embodiments of the Invention

1. A pharmaceutical composition comprising a ribavirin compound for treating an individual having a disease susceptible to treatment with the ribavirin compound and a negative test for at least one ribavirin-induced anemia (RIA) marker,

wherein the RIA marker is selected from the RIA genetic markers in Table 1, or

wherein the RIA marker is normal ITPA activity.

2. Use of a ribavirin compound in the manufacture of a medicament for treating an individual having a disease susceptible to treatment with the ribavirin compound and a negative test for at least one ribavirin-induced anemia (RIA) marker,

wherein the RIA marker is selected from the RIA markers in Table 1, or

wherein the RIA marker is normal ITPA activity.

3. A drug product which comprises a pharmaceutical composition and prescribing information,

wherein the pharmaceutical composition comprises a ribavirin compound and the prescribing information comprises a pharmacogenetic indication,
wherein the pharmacogenetic indication comprises the treatment of a disease susceptible to treatment with the ribavirin compound in patients who test negative for at least one ribavirin-induced anemia (RIA) marker, wherein the RIA marker is selected from the RIA markers in Table 1, or

wherein the RIA marker is normal ITPA activity.

4. A method of testing an individual for the presence or absence of at least one ribavirin-induced anemia (RIA) marker, the method comprising obtaining a nucleic acid sample from the individual and assaying the nucleic acid sample to determine the individual's genotype at a polymorphic site (PS) in Table 1, wherein if the individual is heterozygous or homozygous for the anemia allele for said PS, then the RIA marker is present and if the individual is homozygous for the other allele for said PS, then the RIA marker is absent.
5. The method of embodiment 4, which further comprises generating a test report that indicates the individual's genotype at said PS.
6. A method of testing an individual for the presence of an RIA marker, the method comprising obtaining a biological sample from the individual and assaying the biological sample for the presence of ITPA with proline at amino acid position 32 (ITPA-Pro32).
7. The method of embodiment 6, wherein the assaying step comprises contacting the biological sample with a monoclonal antibody or binding fragment thereof that specifically binds to ITPA-Pro32.
8. The method of embodiment 6, wherein the assaying step comprises contacting the biological sample with each of (1) a monoclonal antibody that specifically binds to ITPA-Pro32, or a binding fragment thereof, and (2) a monoclonal antibody that specifically binds to ITPA-Thr32 or a binding fragment thereof.
9. A method of selecting a therapy for treating an individual having a disease susceptible to treatment with a ribavirin compound, comprising obtaining the individual's genotype at a polymorphic site (PS) selected from the polymorphic sites in Table I and selecting a therapy based on the obtained genotype,

wherein if the individual is heterozygous or homozygous for the anemia allele at the selected PS, then the selected therapy:

(a) comprises administering the ribavirin compound at the dose recommended for the disease in combination with an agent that counteracts ribavirin-induced anemia,

(b) comprises administering the ribavirin compound at a dose lower than the dose recommended for the disease, or

(b) excludes treatment with the ribavirin compound, and

wherein if the individual is homozygous for the other allele at the selected PS, the selected therapy comprises:

(a) administering the ribavirin compound at the dose recommended for the disease or

(b) administering the ribavirin compound at a dose higher than the dose recommended for the disease and monitoring the individual for anemia.

10. A screening method for selecting individuals for initial treatment or continued treatment with ribavirin compound from a group of individuals having a disease susceptible to treatment with the ribavirin compound, comprising testing each member of the disease group for the presence of at least one ribavirin-induced anemia (RIA) marker and excluding from treatment all individuals testing positive for the RIA marker, wherein a positive test for the RIA marker is a heterozygous genotype or a homozygous genotype for the anemia allele for at least one polymorphic site (PS) selected from the polymorphic sites in Table 1.
11. A kit for testing an individual having a disease susceptible to treatment with a ribavirin compound for the presence or absence of a ribavirin-induced anemia (RIA) marker, wherein the kit comprises a set of oligonucleotides designed to genotype at least one polymorphic site (PS) selected from the group of polymorphic sites in Table 1.
12. The kit of embodiment 11, wherein the oligonucleotides are allele specific oligonucleotide (ASO) probes.
13. The kit of embodiment 11 or 12, wherein the oligonucleotides are immobilized on a solid surface.
14. The pharmaceutical composition, use, drug product, method or kit of any of embodiments 1 to 13, wherein the RIA marker is selected from the homozygous RIA markers in Table 1.
15. A method of predicting whether an individual is at risk for severe anemia if treated with a ribavirin compound, the method comprising obtaining an erythrocyte sample from the individual and measuring the ITPA activity in the sample, wherein if the measured ITPA activity is lower than normal then the prediction is that the individual is not likely to experience severe anemia upon treatment with the ribavirin compound, and if the measured ITPA activity is normal or higher than normal, then the prediction is that the individual is likely to experience severe anemia upon treatment with the ribavirin compound.
16. The pharmaceutical composition, use, drug product, method or kit of any of embodiments 1 to 15, wherein the disease susceptible to treatment with the ribavirin compound is a viral infection.
17. The pharmaceutical composition, use, drug product, method or kit of embodiment 16, wherein the viral infection is chronic infection with a hepatitis B virus (HBV) or a hepatitis C virus (HCV).
18. The pharmaceutical composition, use, drug product, method or kit of embodiment 17, wherein the hepatitis C virus is HCV genotype 1.
19. The pharmaceutical composition, use, drug product, method or kit of any of the preceding embodiments, wherein the ribavirin compound is ribavirin or a ribavirin prodrug.
20. The pharmaceutical composition, use, drug product, method or kit of embodiment 19, wherein the ribavirin prodrug is taribavirin.
21. The pharmaceutical composition, use, drug product, method or kit of any of the preceding embodiments, wherein the IFN-α is formulated for parenteral administration.
22. A method of predicting whether an individual chronically infected with HCV will develop anemia in response to a combination therapy comprising an interferon alpha (IFN-α) protein and ribavirin, the method comprising:

obtaining a nucleic acid sample from the individual;

assaying the nucleic acid sample to determine the patient's genotype for at least one polymorphic site (PS) in Table 1; and

making a prediction based on the determined genotype,

wherein If the patient's genotype is heterozygous or homozygous for the anemia allele, then the prediction is that the individual is likely to develop anemia, and if the patient's genotype is homozygous for the other allele, then the prediction is that the individual is not likely to develop anemia.

23. A method of treating an individual for chronic infection with HCV, which comprises:

obtaining the individual's genotype for at least one polymorphic site (PS) in Table 1 and

prescribing a treatment regimen based on the obtained genotype,

wherein If the genotype is heterozygous or homozygous for the anemia allele, then the treatment regimen comprises:

(a) administering to the individual an interferon alpha (IFN-α) protein in combination with ribavirin and at least one agent that counteracts ribavirin-induced anemia; or

(b) administering to the individual an interferon alpha (IFN-α) protein in combination with at least one antiviral agent that is not a ribavirin compound; or

(c) administering to the individual a combination of at least two antiviral agents, neither of which is an interferon alpha protein or a ribavirin compound.

24. The method of embodiment 23, wherein the at least one antiviral agent is an HCV protease inhibitor.
25. The method of embodiment 24, wherein the combination of at least two antiviral agents comprises an HCV protease inhibitor and an HCV polymerase inhibitor.
26. The method of embodiment 24, wherein the HCV protease inhibitor is boceprevir, narlaprevir or telaprevir.
27. The method of any of embodiments 22 to 26, wherein the IFN-α protein is a pegylated interferon alpha-2a protein or an albumin-interferon alpha-2a fusion protein.
28. The method of embodiment 27, wherein the IFN-α protein is PEGASYS® (peginterferon alfa-2a) or a biosimilar thereof.
29. The method of any of embodiments 22 to 26, wherein the IFN-α protein is a pegylated interferon alpha-2b or an albumin-interferon alpha-2b fusion protein.
30. The method of embodiment 29, wherein the IFN-α protein is PegIntron® (peginterferon alfa-2b) or a biosimilar thereof.
31. The pharmaceutical composition, use, drug product, method or kit of any of the preceding embodiments, wherein the individual is self-identified as Caucasian, African American, Hispanic or Asian.
32. The pharmaceutical composition, use, drug product, method or kit of any of the preceding embodiments, wherein the individual is self-identified as Caucasian.
33. The pharmaceutical composition, use, drug product, method or kit of any of the preceding embodiments, wherein the RIA marker is selected from the group consisting of: an A/A genotype at rs6051702, a C/C genotype at rs1127354, an A/A genotype at rs7270101 or normal ITPA activity.
34. The pharmaceutical composition, use, drug product, method or kit of any of the preceding embodiments, wherein the RIA marker is an A/A genotype at rs6051702.
35. The pharmaceutical composition, use, drug product, method or kit of any of the preceding embodiments, wherein the RIA marker is an A/C genotype or a C/C genotype at rs1127354.
36. The pharmaceutical composition, use, drug product, method or kit of any of the preceding embodiments, wherein the RIA marker is an A/C genotype or an A/A genotype at rs7270101.
37. The pharmaceutical composition, use, drug product, method or kit of any of the preceding embodiments, wherein the RIA marker is an A/C genotype at each of rs1127354 and rs7270101.
38. The pharmaceutical composition, use, drug product, method or kit of any of embodiments 1 to 33, wherein the RIA marker is normal ITPA activity.
39. The pharmaceutical composition, use, drug product, method or kit of any of embodiments 1 to 32, wherein the RIA marker is an A/A genotype at the rs6051702 PS if the individual is self-identified as Caucasian, an A/A genotype at rs3810560 PS if the individual is self-identified as African-American, or a T/T genotype at rs11697114 if the individual is self-identified as Hispanic.
40. The kit of embodiment 13, wherein each of the oligonucleotides is immobilized on a separate silica bead.

### EXAMPLES

The following examples are provided to more clearly describe the present invention and should not be construed to limit the scope of the invention.

### Example 1

### Identification of Single Nucleotide Polymorphisms (SNPs) Associated with Ribavirin-Induced Anemia

In order to identify genetic contributions to treatment response, the inventors carried out a genome-wide association study on genomic samples obtained from the IDEAL study, the design of which was reported in McHutchison et al., J. Viral Hepatol., Vol. 15, No. 7, July 2008, pp. 475-481). Briefly, in the IDEAL study, treatment-naive patients chronically infected with HCV genotype 1 were randomized (1:1:1) to receive one of the following 48-week treatment regimens: peginterferon alfa-2b (PEG2b) at 1.5 mcg/kg/week plus ribavirin (RBV); PEG2b at 1.0 mcg/kg/week plus RBV; or peginterferon alfa-2a (PEG2a) at 180 meg/week+RBV. In the PEG2b regimens, patients weighing 40-65 kg received 800 mg/day RBV; patients weighing more than 65 and up to 85 kg received 1000 mg/day RBV; patients weighing more than 85 and up to 105 kg received 1200 mg/day RBV; and patients weighing more than 105 kg received 1400 mg/day RBV). In the PEG2a regimen patients weighing <75 kg received 1000 mg/day of RBV while patients weighing ≧75 kg received 1200 mg/day of RBV. Hemoglobin values were measured at baseline (before the first dose of treatment), at week 2, 4, 8, 12, and then every 6 weeks up to treatment completion (48 weeks total). Follow-up measures were obtained at 4, 12 and 24 weeks post-treatment.

For the genome-wide analysis of RBV-induced anemia, the inventors selected week 4 of therapy as the timepoint for evaluating genetic contribution to three clinical phenotypes: i) absolute reduction in Hb; ii) reduction of Hb≧3 g/dL; iii) reduction of Hb to a level≦10 g/dL. By week 4, significant anemia had occurred, but growth factor therapy had not been instituted. Excluded from this analysis were patients who were <80% adherent to either Peg-IFN or RBV to week 4 (N=95), and patients for whom Hb data was missing at week 4 (N=21). The clinical characteristics of the study population are shown below:

TABLE 4
Clinical characteristics of the study population

| | Populations | | |
|---|---|---|---|
| | European | African | |
| | Americans | Americans | Hispanics |
| N | 988 | 198 | 100 |
| Sex (F/M) | 378/610 | 78/120 | 36/64 |
| Age (yrs) | 47.3 (7.4) | 49.7 (6.6) | 44.8 (9.3) |
| BMI (kg/m²) | 27.9 (4.5) | 29.7 (5.0) | 29.3 (5.4) |
| Baseline weight (kg) | 83.3 (16.1) | 88.7 (14.3) | 83.0 (16.7) |
| Baseline liver fibrosis stage | | | |
| (n, %) | | | |
| Minimal (F0-2) | 876 (88.7%) | 182 (91.9%) | 86 (86.0%) |
| Advanced (F3-4) | 112 (11.3%) | 16 (8.1%) | 14 (14.0%) |
| Baseline hemoglobin value | 15.1 (1.2) | 14.6 (1.2) | 15.2 (1.3) |
| (g/dL) | | | |
| Initial Ribavirin dose (n, %) | | | |
| 800 mg | 88 (8.9%) | 4 (2.0%) | 6 (6.0%) |
| 1000 mg | 377 (38.2%) | 63 (31.8%) | 41 (41.0%) |
| 1200 mg | 460 (46.6%) | 117 (59.1%) | 45 (45.0%) |
| 1400 mg | 63 (6.4%) | 14 (7.1%) | 8 (8.0%) |
| Peg-interferon treatment | | | |
| PegIFN2a | 330 (33.4%) | 66 (33.3%) | 31 (31.0%) |
| PegIFN2b 1.0 | 333 (33.7%) | 69 (34.9%) | 32 (32.0%) |
| PegIFN2b 1.5 | 325 (32.9%) | 63 (31.8%) | 37 (37.0%) |

BMI, body mass index.
Fibrosis was scored by METAVIR stage on a baseline centrally evaluated liver biopsy.
Data are mean (SD) unless otherwise indicated.

Genomic samples from 1286 individuals were genotyped using the Human610-quad BeadChip from Illumina® (San Diego, Calif.), which contains about 600,000 tagging SNPs derived from phase II HapMap data (HumanHap 610 quad V 1.0). A series of quality control steps resulted in 565,759 polymorphisms for the association tests.

The primary association tests involved single-marker genotype trend tests of association between each single nucleotide polymorphism (SNP) and the Hb phenotypes, using linear and logistic regression models implemented in the PLINK software (Purcell, S. et al. Am J Hum Genet. 81 (2007)) with corrections for a number of covariates, including age, gender, weight, fibrosis severity on pretreatment liver biopsy, baseline hemoglobin level, as well as the dose of RSV and the type and dose of PegIFN that were administered in the study. Separate analyses were run in the 3 ethnic groups. To control for the possibility of spurious associations resulting from population stratification, a modified EIGENSTRAT method (Price, A. L. et al. Nat Genet. 38, 904-9 (2006)) was used to correct for population ancestry axes within each ethnic population. Significance was assessed with a Bonferroni correction (P cutoff-8.8×10⁻⁸). Table 5 below lists the 20 SNPs showing the strongest association after combination of P values by the Stouffer's weighted Z-method, together with their P values in the 3 ethnic populations. All of the SNPs map to 20p13, except rs10159477, which is an intronic variant of the HK1 gene on chromosome 10.

TABLE 5
Results of the GWAS of determinants of absolute hemoglobin
reduction in HCV G1 patients after 4 weeks of treatment with
ribavirin and peginterferon alfa-2a or peginterferon alfa-2b.

| | Populations | | | |
|---|---|---|---|---|
| | European | African | | |
| SNP | American | Americans | Hispanics | Combined |
| rs6051702 | 1.15E−45 | 0.19 | 9.5E−03 | 1.00E−46 |
| rs3310 | 1.15E−45 | 0.25 | 6.3E−03 | 1.29E−46 |
| rs965469 | 1.95E−45 | 0.23 | 1.5E−02 | 3.00E−46 |
| rs6051762 | 1.04E−44 | 0.62 | 2.8E−02 | 1.33E−44 |
| rs6051841 | 6.57E−38 | 0.03 | 4.2E−02 | 1.25E−39 |
| rs6051693 | 1.91E−32 | 0.25 | 6.5E−03 | 1.62E−33 |
| rs11697114 | 2.90E−21 | 0.03 | 2.1E−04 | 1.16E−23 |
| rs6115892 | 1.01E−21 | 0.24 | 4.6E−01 | 5.06E−22 |
| rs6115865 | 4.40E−21 | 0.41 | 2.4E−01 | 2.74E−21 |
| rs6051855 | 3.13E−18 | 0.39 | 3.7E−01 | 2.05E−18 |
| rs11697620 | 2.10E−16 | 0.28 | 1.5E−02 | 2.55E−17 |
| rs2295547 | 8.89E−16 | 0.53 | 9.7E−02 | 3.02E−15 |
| rs8120592 | 2.78E−12 | 0.67 | 3.6E−03 | 2.13E−12 |
| rs3827075 | 1.13E−10 | 0.21 | 9.3E−01 | 6.61E−11 |
| rs2326084 | 2.31E−08 | 0.14 | 3.3E−03 | 1.67E−09 |
| rs1207 | 1.32E−08 | 0.42 | 4.7E−01 | 1.03E−07 |
| rs2295545 | 3.35E−07 | 0.74 | 8.2E−02 | 1.84E−07 |
| rs10159477 | 5.28E−07 | 0.44 | 8.5E−02 | 1.85E−07 |
| rs6076519 | 7.26E−08 | 0.64 | 8.1E−01 | 2.71E−07 |
| rs6051689 | 2.73E−06 | 0.11 | 3.4E−02 | 3.52E−07 |

The SNPs showing the strongest association with Hb reduction in each of the three ethnic groups are set forth in Table 6 below, together with their P values in the different ethnic groups.

TABLE 6
SNPs associated with absolute reduction in hemoglobin in
HCV G1 patients after 4 weeks of treatment with Peg-IFN
alfa-2a or alfa-2b/RBV.

| | | P value in | P value in | |
|---|---|---|---|---|
| | | European | African | P value in |
| | Most associated | Americans | American | Hispanics |
| SNP | SNP in: | (N = 988) | (N = 198) | (N = 100) |
| rs6051702 | European Americans | 1.1 × 10⁻⁴⁵ | 1.9 × 10⁻¹ | 9.5 × 10⁻³ |
| rs3810560 | African Americans | 2.6 × 10⁻² | 1.2 × 10⁻⁴ | 3.0 × 10⁻¹ |
| rs1169711 | Hispanics | 2.0 × 10⁻⁶ | 2.8 × 10⁻² | 2.1 × 10⁻⁴ |

The independence of the top association signals in the European American population were tested using nested linear regression models, in which individual SNPs were added after inclusion of rs6051702, the most associated variant. Additional independent associations were observed for rs2295547 (P=1.4×10⁻⁹) and rs6051855 (P=2.3×10⁻⁴), suggesting the existence of several causal sites resulting in synthetic associations.

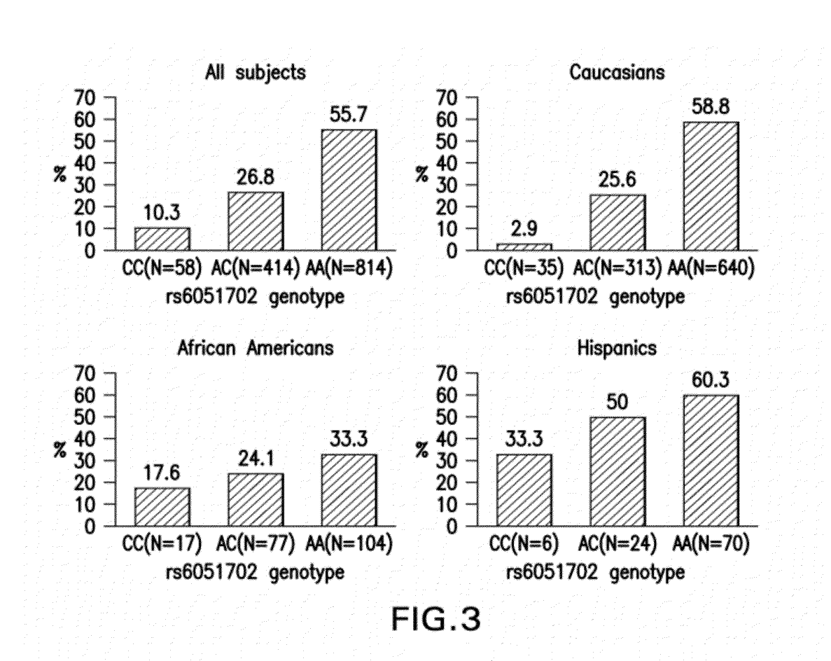

The association of the rs6051702 SNP with RBV-induced anemia was confirmed in a case-control analysis that compared subjects with more or less than 3 g/dL decrease in Hb after 4 weeks of treatment. As evident from the data shown in FIG. 3, rs6051702 SNP again showed the strongest association signal in European Americans: only 2.9% of patients with the CC genotype showed a decrease in Hb of at least 3 g/dL, while 58.8% of patients that are homozygous for the major allele AA reached this threshold. Concordantly, none of the CC patients had Hb concentrations<10 g/dL at week 4, whereas 13% of patients with the AA genotype were documented to develop severe anemia (data not shown).

### Example 2

### Identification of Candidate Causal Polymorphisms for Ribavirin-Induced Anemia

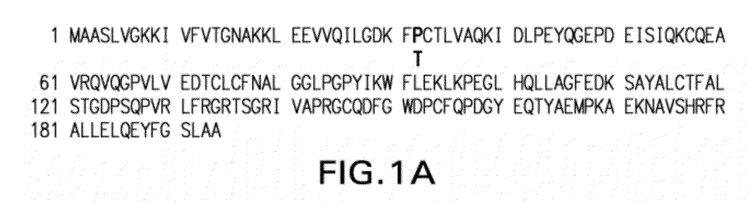

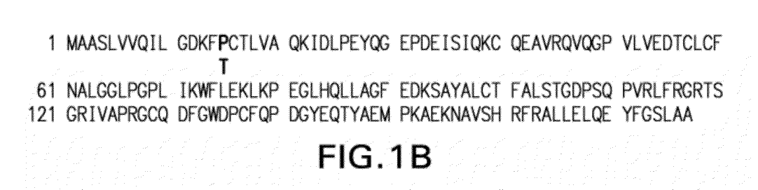

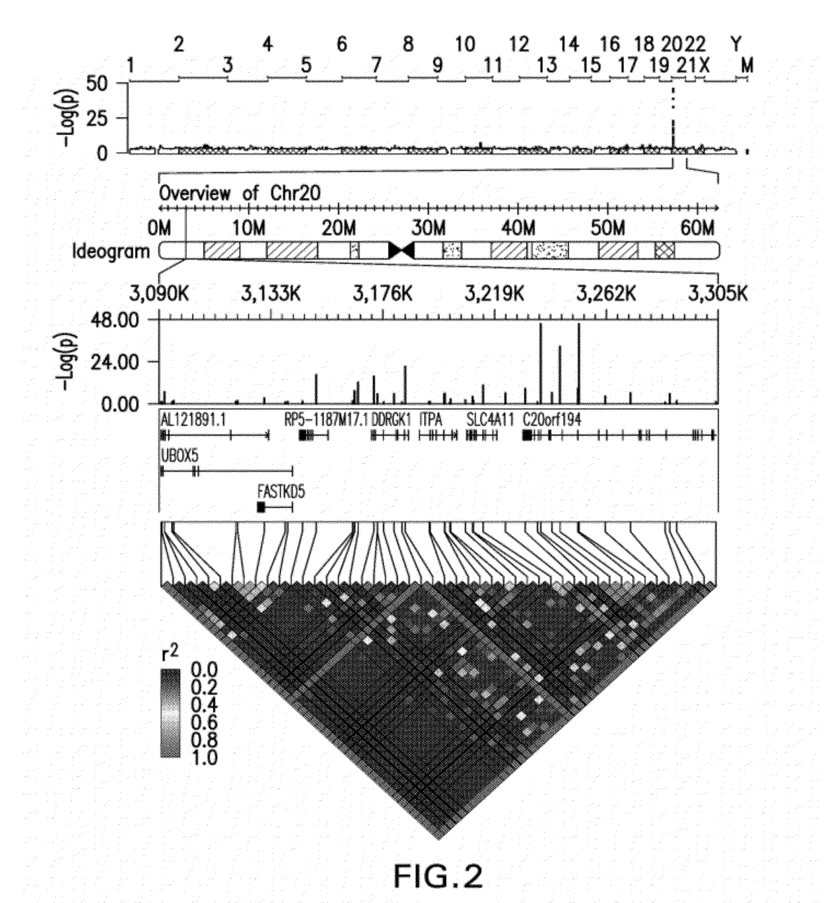

A total of 15 SNPs showed a genome-wide significant association with quantitative Hb reduction in the combined analysis: these SNPs were spread over a 250 kb region that contains 5 different protein-coding genes (FIG. 1). One of these genes is the ITPA gene. Since two SNPs in the ITPA gene (rs1127354, resulting in a P32T amino acid variation, and rs7270101, a splicing-altering SNP located in the second intron) have been functionally associated with ITPA deficiency and increased thiopurine toxicity, the inventors used HapMap data for CEPH parents (The International HapMap Consortium, Nature 437:1299-1320 (2005)) to investigate the degree of linkage disequilibrium between these ITPA SNPs and the rs6051702 SNP. The analysis included 56 CEU parents with complete genotype data for the rs1127354, rs7270101 and rs6051702 SNPs. They found that each of the ITPA alleles that are associated with ITPA deficiency are preferentially associated with the rs6051702 C allele, which was associated with less hemoglobin reduction.

To further evaluate the potential functional role of ITPA activity in RBV-induced anemia, the inventors collapsed the two low activity alleles into a new variable and tested this variable for LD with all of the HapMap SNPs located in the surrounding 1 Mb region. The highest r² was 0.65, which was observed for the rs6051702 SNP and 26 other SNPs in this region (see Table 7).

TABLE 7
SNPs in LD with combined variable of two ITPA low activity alleles.

| | r² with | D′ with |
|---|---|---|
| SNP | combined variable | combined |
| rs6051702 | 0.649 | 0.828 |
| rs3310 | 0.649 | 0.828 |
| rs7274193 | 0.649 | 0.828 |
| rs2236094 | 0.649 | 0.828 |
| rs6051708 | 0.649 | 0.828 |
| rs6051790 | 0.649 | 0.828 |
| rs6037553 | 0.649 | 0.828 |
| rs6139064 | 0.649 | 0.828 |
| rs4611719 | 0.649 | 0.828 |
| rs2236123 | 0.649 | 0.828 |
| rs2236118 | 0.649 | 0.828 |
| rs6139068 | 0.649 | 0.828 |
| rs2236122 | 0.649 | 0.828 |
| rs2236104 | 0.649 | 0.828 |
| rs6037567 | 0.649 | 0.828 |
| rs6051716 | 0.649 | 0.828 |
| rs6051807 | 0.649 | 0.828 |
| rs6051753 | 0.649 | 0.828 |
| rs6051764 | 0.649 | 0.828 |
| rs1040726 | 0.649 | 0.828 |
| rs2281500 | 0.649 | 0.828 |
| rs965469 | 0.649 | 0.828 |
| rs6037554 | 0.649 | 0.828 |
| rs2236089 | 0.649 | 0.828 |
| rs7270135 | 0.649 | 0.828 |
| rs6037560 | 0.649 | 0.828 |
| rs6051713 | 0.649 | 0.828 |

### Example 3

### Association of ITPA Deficiency with Protection Against Ribavirin-Induced Anemia

The results described in Example 2 suggested the possibility that low ITPA activity confers protection against ribavirin-induced hemolytic anemia. To test this possibility, the inventors sequenced the entire coding region of the ITPA gene in genomic samples from 168 patients in the study population samples and genotyped the rs1127354 and rs7270101 SNPs in the entire study population and analyzed the various genotypes for association with the rs6051702 C allele, identified in the GWAS, and for independent association with treatment-induced Hb reduction.

The sequencing revealed no other obvious reduced function mutations that could contribute to the association signal (data not shown). However, when the low activity alleles of the rs1127354 and rs7270101 ITPA SNPs were incorporated into a regression model, they entirely explained the association observed in the GWAS described in Example 1 (data not shown). Also, in the HCV patients of European American ancestry, the two low activity ITPA alleles were found almost exclusively on chromosomes that also carry the C allele of the rs6051702 PS that was associated with less Hb reduction during Peg-IFN/RBV therapy. These data are shown in Table 8 below.

TABLE 8
Co-segregation of ITPA low activity alleles with the rs6051702 C allele in European
Americans chronically infected with HCV genotype 1.

| | | rs1127354 | | rs7270101 | | |
|---|---|---|---|---|---|---|
| | | (94C > A = P32T) | | (IVS2 + 21A > C) | | At least one ITPA |
| rs6051702 | N | Het | Homo | Het | Homo | deficiency allele |
| CC | 35 | 11 (31%) | 8 (23%) | 16 (46%) | 9 (26%) | 34 (97.1%) |
| AC | 311 | 87 (28%) | 2 (1%) | 172 (56%) | 7 (2%) | 262 (84.2%) |
| AA | 640 | 32 (5%) | 0 (0%) | 21 (3%) | 0 (0%) | 52 (8.1%) |
| All | 986 | 130 (13%) | 10 (1%) | 209 (21%) | 16 (2%) | 348 (35.3%) |

Each of these ITPA low activity alleles is also independently associated with protection against ribavirin-induced anemia in the study population as shown in Table 9 below.

TABLE 9
Association between low ITPA activity alleles and protection against Hb reduction in chronically infected HCV genotype 1 patients
treated with Peg-IFN/RBV therapy.

| ITPA low | European Americans | | | African American | | | Hispanics | | | All (combined) | | |
|---|---|---|---|---|---|---|---|---|---|---|---|---|
| activity | MAF | | Ind. P | | | Ind. P | MAF | | Ind. P | | | Ind. P |
| alleles | % | P value | value | MAF % | P value | value | % | P value | value | MAF % | P value | value |
| rs1127354 | 7.6 | 4.6 × 10⁻⁵² | 2.3 × 10⁻⁶⁸ | 4.6 | 2.7 × 10⁻⁷ | 5.1 × 10⁻⁷ | 4.0 | 1.2 × 10⁻³ | 5.6 × 10⁻⁵ | 6.9 | 1.7 × 10⁻⁵⁸ | 5.9 × 10⁻²⁶ |
| rs7270101 | 12.3 | 6.8 × 10⁻²² | 3.6 × 10⁻³⁸ | 7.9 | 3.0 × 10⁻⁵ | 6.6 × 10⁻⁵ | 8.0 | 3.8 × 10⁻⁴ | 1.9 × 10⁻⁵ | 11.2 | 8.5 × 10⁻⁷⁶ | 2.6 × 10⁻⁴³ |

Ind. P value: Independent P values were calculated in models in which the other functional variant was already included.
Combined P values for all three populations were obtained using the Stouffer's weight Z-method (Whitlock MC. Combining probability from independent tests: the weighted Z-method is superior to Fisher's approach. Journal of Evolutionary Biology 2005; 18: 1368-1373).
MAF: minor allele frequency.

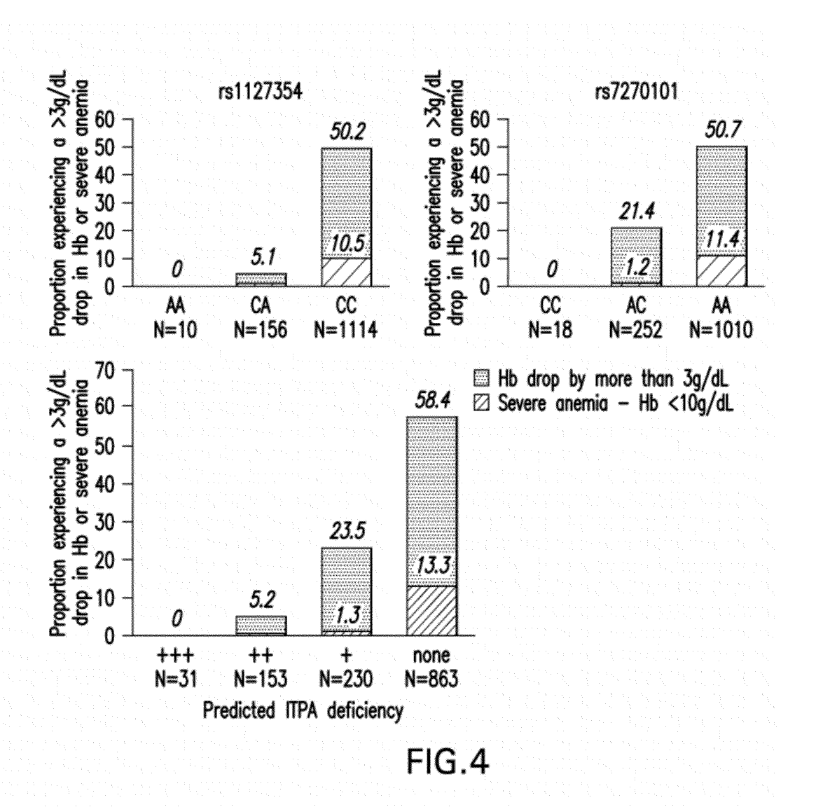

The clinical relevance of these variants was assessed by inspecting the proportion of patients suffering moderate or severe anemia (defined as a decrease in Hb of ≧3 g/dL or Hb levels≦10 g/dL, respectively) as a function of the individual genotypes or of the degree of ITPA deficiency estimated from Shipkova, M. et al., Clin Chem. 52:240-247 (2006): in comparison to wild type homozygous, ITPA activity decreased to 60% with rs7270101 heterozygosity; to 30% with rs1127354 heterozygosity or rs7270101 homozygosity; and to a very low residual activity with combined heterozygosity or rs1127354 homozygosity. The data are shown in FIG. 4. In 184 patients predicted to have less than one third of normal ITPA enzymatic activity, none was observed to have severe anemia and only 4.3% had moderate anemia (lower graph, left two bars). On the other hand, of the 863 patients with predicted “normal” ITPA function, 13.3% suffered severe anemia (i.e., Hb decrease to ≦10 g/dL) and 58.4% developed moderate anemia (Hb decrease of ≧3 g/dL).

In conclusion, the identification of inosine triphosphatase deficiency as a major projective factor against RBV-induced hemolytic anemia provides the basis for the detection of ITPA deficiency alleles or measurement of ITPA activity in pharmacogenetic diagnostic methods and products. Also, since ITPA deficiency appears to be a benign condition, it may be possible to protect against RBV induced anemia by pharmacological intervention against ITPA.

1-20. (canceled)

21. A drug product which comprises a pharmaceutical composition and prescribing information,
wherein the pharmaceutical composition comprises a ribavirin compound and the prescribing information comprises a pharmacogenetic indication, wherein the pharmacogenetic indication comprises the treatment of a disease susceptible to treatment with the ribavirin compound in patients who test negative for at least one ribavirin-induced anemia (RIA) marker, wherein the RIA marker selected from the RIA markers in the Table below:

| | | | Hetero- | |
|---|---|---|---|---|
| | | Anemia | zygous | Homozygous |
| PS | SNP | Allele | RIA Marker | RIA Marker |
| rs6051702 | A/C | A | A/C | A/A genotype |
| | | | genotype | |
| rs3810560 | A/G | A | A/G | A/A genotype |
| | | | genotype | |
| rs11697114 | T/C | T | T/C | T/T genotype |
| | | | genotype | |
| rs3310 | T/C | C | T/C | C/C genotype |
| | | | genotype | |
| rs965469 | T/C | T | T/C | T/T genotype |
| | | | genotype | |
| rs6051762 | T/C | T | T/C | T/T genotype |
| | | | genotype | |
| rs6051841 | T/C | T | T/C | T/T genotype |
| | | | genotype | |
| rs6051693 | T/G | T | T/G | T/T genotype |
| | | | genotype | |
| rs6115892 | T/C | C | T/C | C/C genotype |
| | | | genotype | |
| rs6115865 | T/C | C | T/C | C/C genotype |
| | | | genotype | |
| rs6051855 | T/C | T | T/C | T/T genotype |
| | | | genotype | |
| rs11697620 | A/G | A | A/G | A/A genotype |
| | | | genotype | |
| rs2295547 | A/C | C | A/C | C/C genotype |
| | | | genotype | |
| rs8120592 | T/C | C | T/C | C/C genotype |
| | | | genotype | |
| rs3827075 | A/C | C | A/C | C/C genotype |
| | | | genotype | |
| rs2326084 | A/C | A | A/C | A/A genotype |
| | | | genotype | |
| rs1207 | T/C | T | T/C | T/T genotype |
| | | | genotype | |
| rs2295545 | T/C | C | T/C | C/C genotype |
| | | | genotype | |
| rs10159477 | T/C | T | T/C | T/T genotype |
| | | | genotype | |
| rs6076519 | T/C | C | T/C | C/C genotype |
| | | | genotype | |
| rs6051689 | A/G | G | A/G | G/G genotype |
| | | | genotype | |
| rs1127354 | C/A | C | A/C | C/C genotype |
| | | | genotype | |
| rs7270101 | A/C | A | A/C | A/A genotype |
| | | | genotype | |
| rs7274193 | C/T | C | C/T | C/C genotype |
| | | | genotype | |
| rs2236094 | G/C | G | G/C | G/G genotype |
| | | | genotype | |
| rs6051708 | T/C | T | T/C | T/T genotype |
| | | | genotype | |
| rs6051790 | C/T | C | C/T | C/C genotype |
| | | | genotype | |
| rs6037553 | A/G | A | A/G | A/A genotype |
| | | | genotype | |
| rs6139064 | G/T | G | G/T | G/G genotype |
| | | | genotype | |
| rs4611719 | A/G | A | A/G | A/A genotype |
| | | | genotype | |
| rs2236123 | C/G | C | C/G | C/C genotype |
| | | | genotype | |
| rs2236118 | G/A | G | G/A | G/G genotype |
| | | | genotype | |

or wherein the RIA marker is normal ITPA activity.

22. The drug product of claim 21, wherein the RIA marker is selected from the homozygous RIA markers in the Table, disease susceptible to treatment with the ribavirin compound is a viral infection, and the ribavirin compound is ribavirin or a ribavirin prodrug.

23. The drug product of claim 22, wherein the viral infection is chronic infection with a hepatitis B virus (HBV) or a hepatitis C virus (HCV).

24. The drug product of claim 23, wherein the RIA marker is selected from the group consisting of:
an A/A genotype at rs6051702;
a C/C genotype at rs1127354;
an A/A genotype at rs7270101;
an A/C genotype at each of rs1127354 and rs7270101; and
normal ITPA activity.

25. A method of testing an individual for the presence or absence of at least one ribavirin-induced anemia (RIA) marker, the method comprising:
(a) obtaining a nucleic acid sample from the individual and assaying the nucleic acid sample to determine the individual's genotype at a polymorphic site (PS) in the Table below:

| | | Ane- | | |
|---|---|---|---|---|
| | | mia | | |
| | | Al- | Heterozygous | Homozygous |
| PS | SNP | lele | MA Marker | MA Marker |
| rs6051702 | A/C | A | A/C genotype | A/A genotype |
| rs3810560 | A/G | A | A/G genotype | A/A genotype |
| rs11697114 | T/C | T | T/C genotype | T/T genotype |
| rs3310 | T/C | C | T/C genotype | C/C genotype |
| rs965469 | T/C | T | T/C genotype | T/T genotype |
| rs6051762 | T/C | T | T/C genotype | T/T genotype |
| rs6051841 | T/C | T | T/C genotype | T/T genotype |
| rs6051693 | T/G | T | T/G genotype | T/T genotype |
| rs6115892 | T/C | C | T/C genotype | C/C genotype |
| rs6115865 | T/C | C | T/C genotype | C/C genotype |
| rs6051855 | T/C | T | T/C genotype | T/T genotype |
| rs11697620 | A/G | A | A/G genotype | A/A genotype |
| rs2295547 | A/C | C | A/C genotype | C/C genotype |
| rs8120592 | T/C | C | T/C genotype | C/C genotype |
| rs3827075 | A/C | C | A/C genotype | C/C genotype |
| rs2326084 | A/C | A | A/C genotype | A/A genotype |
| rs1207 | T/C | T | T/C genotype | T/T genotype |
| rs2295545 | T/C | C | T/C genotype | C/C genotype |
| rs10159477 | T/C | T | T/C genotype | T/T genotype |
| rs6076519 | T/C | C | T/C genotype | C/C genotype |
| rs6051689 | A/G | G | A/G genotype | G/G genotype |
| rs1127354 | C/A | C | A/C genotype | C/C genotype |
| rs7270101 | A/C | A | A/C genotype | A/A genotype |
| rs7274193 | C/T | C | C/T genotype | C/C genotype |
| rs2236094 | G/C | G | G/C genotype | G/G genotype |
| rs6051708 | T/C | T | T/C genotype | T/T genotype |
| rs6051790 | C/T | C | C/T genotype | C/C genotype |
| rs6037553 | A/G | A | A/G genotype | A/A genotype |
| rs6139064 | G/T | G | G/T genotype | G/G genotype |
| rs4611719 | A/G | A | A/G genotype | A/A genotype |
| rs2236123 | C/G | C | C/G genotype | C/C genotype |
| rs2236118 | G/A | G | G/A genotype | G/G genotype |

wherein if the individual is heterozygous or homozygous for the anemia allele for said PS, then the RIA marker is present and if the individual is homozygous for the other allele for said PS, then the RIA marker is absent; or
(b) obtaining a biological sample from the individual and assaying the biological sample for the presence of ITPA with proline at amino acid position 32 (ITPA-Pro32).

26. The method of claim 25, wherein the method comprises the steps in part (a) and which further comprises generating a test report that indicates the individual's genotype at said PS.

27. The method of claim 25, wherein the method comprises the steps in part (b) and the assaying step comprises contacting the biological sample with a monoclonal antibody or binding fragment thereof that specifically binds to ITPA-Pro32.

28. The method of claim 27, wherein the assaying step comprises contacting the biological sample with each of (1) a monoclonal antibody that specifically binds to ITPA-Pro32, or a binding fragment thereof, and (2) a monoclonal antibody that specifically binds to ITPA-Thr32 or a binding fragment thereof.

29. The method of claim 25, wherein the RIA marker is selected from the homozygous RIA markers in the Table, the disease susceptible to treatment with the ribavirin compound is a viral infection, and the ribavirin compound is ribavirin or a ribavirin prodrug.

30. The method of claim 29, wherein the viral infection is chronic infection with a hepatitis B virus (HBV) or a hepatitis C virus (HCV).

31. The method of claim 30, wherein the RIA marker is selected from the group consisting of:
an A/A genotype at rs6051702;
a C/C genotype at rs1127354;
an A/A genotype at rs7270101;
an A/C genotype at each of rs1127354 and rs7270101; and
normal ITPA activity.

32. A method of treating an individual for chronic infection with HCV, which comprises:
obtaining the individual's genotype for at least one polymorphic site (PS) in the Table below:

| | | Anemia | Heterozygous | Homozygous |
|---|---|---|---|---|
| PS | SNP | Allele | RIA Marker | RIA Marker |
| rs6051702 | A/C | A | A/C genotype | A/A genotype |
| rs3810560 | A/G | A | A/G genotype | A/A genotype |
| rs11697114 | T/C | T | T/C genotype | T/T genotype |
| rs3310 | T/C | C | T/C genotype | C/C genotype |
| Rs965469 | T/C | T | T/C genotype | T/T genotype |
| Rs6051762 | T/C | T | T/C genotype | T/T genotype |
| Rs6051841 | T/C | T | T/C genotype | T/T genotype |
| Rs6051693 | T/G | T | T/G genotype | T/T genotype |
| Rs6115892 | T/C | C | T/C genotype | C/C genotype |
| Rs6115865 | T/C | C | T/C genotype | C/C genotype |
| Rs6051855 | T/C | T | T/C genotype | T/T genotype |
| Rs11697620 | A/G | A | A/G genotype | A/A genotype |
| Rs2295547 | A/C | C | A/C genotype | C/C genotype |
| Rs8120592 | T/C | C | T/C genotype | C/C genotype |
| Rs3827075 | A/C | C | A/C genotype | C/C genotype |
| Rs2326084 | A/C | A | A/C genotype | A/A genotype |
| Rs1207 | T/C | T | T/C genotype | T/T genotype |
| Rs2295545 | T/C | C | T/C genotype | C/C genotype |
| Rs10159477 | T/C | T | T/C genotype | T/T genotype |
| Rs6076519 | T/C | C | T/C genotype | C/C genotype |
| Rs6051689 | A/G | G | A/G genotype | G/G genotype |
| Rs1127354 | C/A | C | A/C genotype | C/C genotype |
| Rs7270101 | A/C | A | A/C genotype | A/A genotype |
| Rs7274193 | C/T | C | C/T genotype | C/C genotype |
| Rs2236094 | G/C | G | G/C genotype | G/G genotype |
| Rs6051708 | T/C | T | T/C genotype | T/T genotype |
| Rs6051790 | C/T | C | C/T genotype | C/C genotype |
| Rs6037553 | A/G | A | A/G genotype | A/A genotype |
| Rs6139064 | G/T | G | G/T genotype | G/G genotype |
| Rs4611719 | A/G | A | A/G genotype | A/A genotype |
| Rs2236123 | C/G | C | C/G genotype | C/C genotype |
| Rs2236118 | G/A | G | G/A genotype | G/G genotype |

and prescribing a treatment regimen based on the obtained genotype, wherein if the genotype is heterozygous or homozygous for the anemia allele, then the treatment regimen comprises:
(a) administering to the individual an interferon alpha (IFN-α) protein in combination with ribavirin and at least one agent that counteracts ribavirin-induced anemia; or
(b) administering to the individual an interferon alpha (IFN-α) protein in combination with at least one antiviral agent that is not a ribavirin compound; or
(c) administering to the individual a combination of at least two antiviral agents, neither of which is an interferon alpha protein or a ribavirin compound.

33. The method of claim 32, wherein the at least one antiviral agent is an HCV protease inhibitor.

34. The method of claim 32, wherein the combination of at least two antiviral agents comprises an HCV protease inhibitor and an HCV polymerase inhibitor.

35. The method of claim 32, wherein the HCV protease inhibitor is boceprevir, narlaprevir or telaprevir.

36. The method of claim 32, wherein the IFN-α protein is a pegylated interferon alpha-2a protein, an albumin-interferon alpha-2a fusion protein, a pegylated interferon alpha-2b or an albumin-interferon alpha-2b fusion protein.

37. The method of claim 36, wherein the IFN-α protein is a pegylated interferon alpha-2b.

38. The method of claim 32, wherein the individual is self-identified as Caucasian, African American, Hispanic or Asian.

39. The method of claim 32, wherein the RIA marker is selected from the group consisting of:
an A/A genotype at rs6051702;
a C/C genotype at rs1127354;
an A/A genotype at rs7270101;
an A/C genotype at each of rs1127354 and rs7270101; and
normal ITPA activity.

40. The method of claim 32, wherein the RIA marker is an A/A genotype at the rs6051702 PS if the individual is self-identified as Caucasian, an A/A genotype at rs3810560 PS if the individual is self-identified as African-American, or a T/T genotype at rs11697114 if the individual is self-identified as Hispanic.
